(19) United States
(12) Patent Application Publication
(10) Pub. No.: US 2019/0376067 A1
(43) Pub. Date: Dec. 12, 2019

(54) COMPOSITIONS, METHODS AND USES FOR MULTIPLEXED TRACKABLE GENOMICALLY-ENGINEERED POLYPEPTIDES

(71) Applicant: The Regents of the University of Colorado, a Body Corporate, Denver, CO (US)

(72) Inventors: Ryan T. Gill, Boulder, CO (US); William Grau, Denver, CO (US)

(21) Appl. No.: 16/485,333

(22) PCT Filed: Feb. 13, 2018
(86) PCT No.: PCT/US18/18073

§ 371 Date: Aug. 12, 2019

Related U.S. Application Data
(60) Provisional application No. 62/458,483, filed on Feb. 13, 2017.

(51) Int. Cl. C12N 15/52; C12N 15/70; G16B 25/00; C40B 50/10; C40B 40/08; C12N 15/10

(52) U.S. Cl. CPC C12N 15/52; C12N 15/70; G16B 25/00; C12N 9/10; C40B 40/08; C12N 15/1072; C12N 2310/20; C40B 50/10

(57) ABSTRACT

Embodiments herein concern compositions, methods, systems and uses for in vivo selection of optimum target proteins of use in designing genomically-engineered cells or organisms. Some embodiments relate to compositions and methods for generating constructs mimicking benefits of megasynthases in a non-natural organism or cell of use in systems and methods disclosed herein. Yet other embodiments relate to compositions and methods for generating agents using constructs disclosed herein of use in treating genetically-linked conditions.

### PRIORITY

This PCT application claims the benefit under 35 USC § 119(e) of provisional U.S. patent application Ser. No. 62/458,483 filed on Feb. 13, 2017, which is incorporated herein by reference in its entirety for all purposes.

### STATEMENT REGARDING GOVERNMENT FUNDING

This invention was made with government support under grant number DE-SC0008812 awarded by the U.S. Department of Energy. The government has certain rights in the invention.

### FIELD

Embodiments herein report compositions, systems, methods, and uses for generating comprehensive in vivo libraries related to genetic variations for producing target molecules such as proteins, peptides, polypeptides, target agents, small molecules and chemicals. In certain embodiments, target molecules can be prokaryotic or eukaryotic target polypeptides, peptides, proteins or other agents of use in a variety of applications. In other embodiments, target molecules can be generated related to producing biofuels, biotech agents and biopharmaceutical agents or chemicals of use for small or large scale production or screening. Some embodiments of the present disclosure include creating genetic constructs using conserved domains (e.g. catalytic domains) associated with other conserved domains (e.g. catalytic domains) capable of generating a target molecule(s) of interest. Other embodiments include methods of generating such constructs. Yet other embodiments herein report systems that can include computer generated/created or analyzed platform technology construct systems having input and/or output parameters and/or methodologies for assessing and compiling certain target molecule pools. In some embodiments, constructs can include catalytic domains derived from megasynthases, rearranged in a non-naturally occurring order linked together to form constructs for producing target molecules and mixtures of related target molecules.

### REFERENCE TO SEQUENCE LISTING

This application contains a Sequence Listing submitted via EFS-web and is hereby incorporated by reference in its entirety for all purposes. The ASCII copy, created for this application is named 20180213_466888.61_SEQUENCE_LISTING_ST25 and is 2.53 MB in size.

### BACKGROUND

Many natural products are synthesized by elaborate pathways using enzymes, frequently using a particular class of enzymes. Some of these natural products are synthesized by enzymes referred to as megasynthases. Predictable combinatorial biosynthesis of such megasynthases, including using re-programmable megasynthases to produce certain molecules, is of particular interest due to the broad uses of the resultant natural products from these enzymes such as chemicals with pharmaceutical, flavor, and/or fragrance applications.

Combinatorial biosynthesis of megasynthases is a challenge as it requires manipulation of large DNA constructs. A fundamental limitation to synthetic biology and genome engineering practices is the inability to effectively manipulate complex phenotypes, for which the relevant combinatorial mutational space is often much larger than can be searched on laboratory time scales.

Microbial genomes hold the potential for creating extraordinary combinatorial diversity. Searching these variations for specific genetic features that affect pertinent target molecules and traits remains limited by the number of individual variations that can be identified and tested at a time, which is a very small fraction of all possibilities. This issue has been studied at the level of individual mutations, where high-throughput methods for introducing specific mutations in residues and then mapping the effect of such mutations onto target molecule activity are available. Yet other impeding issues are that use of these enzymes (e.g. megasynthases) in non-natural organisms (e.g. bacteria) fail to produce functioning enzymes once combinatorial and genetic manipulations are introduced.

### SUMMARY

Embodiments disclosed herein concern compositions, systems, methods, and uses for generating comprehensive in vivo libraries related to genetic variations for producing target molecules such as proteins, peptides, polypeptides, target agents, small molecules and chemicals. In certain embodiments, target molecules can be prokaryotic or eukaryotic target polypeptides, peptides, proteins or other agents of use in a variety of applications. In certain embodiments, target molecules can be generated related to producing biofuels, biotech agents and biopharmaceutical agents or small molecules or chemicals of use for small or large scale production or screening. Some embodiments of the present disclosure include creating genetic constructs using conserved domains (e.g. catalytic domains) associated with other conserved domains (e.g. catalytic domains) selectively linked to one another that are capable of generating a target molecule(s) of interest. Some embodiments of the present disclosure include creating genetic constructs capable of generating a target molecule or family of related molecules. Other embodiments include methods of generating such constructs.

Other embodiments disclosed herein report systems that can include computer generated/created and/or analyzed platform technology construct systems having input and/or output parameters and/or methodologies for assessing and compiling target molecule pools or families. In some embodiments, these systems can include a computer-readable medium, the computer-readable medium having computer-readable instructions, which, when executed by a computer, cause the computer to carry out a method. In some embodiments, the method can include multiple steps, those steps including (1) receiving a first gene(s) or genetic segment score representing a score of a biological effect or condition due to a genetic variation of a gene or gene segment of a target protein, (2) receiving at least a second gene(s) or genetic score representing a second score of another genetic variation of the target protein, (3) combining the scores; and (4) assigning a combined score related to one or more genetic variations in order to assess a value of the genetic variations related to a trait for the target protein. In other embodiments, the computer-readable medium can further include designing a genomically-engineered organism or cell based on the composite scores for two or more genes or genetic loci. In some embodiments, information related to more than one target gene can be received and assessed by the computer-readable medium.

In certain embodiments, constructs can include catalytic domains of known enzymes rearranged and linked to form non-naturally occurring constructs for producing target molecules and mixtures of related target molecules of use as pharmaceutical agents. In some embodiments, constructs can include catalytic domains derived from megasynthases, rearranged in a non-naturally occurring order linked together to form constructs, often modular megasynthases, for producing target molecules and mixtures of related target molecules.

Other embodiments disclosed herein generally relate to compositions, systems and methods for compiling and assessing mutational libraries of one or more target protein(s). In some embodiments, one or more target proteins can be a prokaryotic protein or a eukaryotic protein. In other embodiments, target proteins and domains thereof of use in constructs of certain embodiments herein can include, but are not limited to, modular megasynthases, polyketide synthases (PKS), non-ribosomal peptide synthases (NRPS), and/or PKS-NRPS hybrids.

Certain embodiments herein concern constructs for compiling an in vivo library of one or more target proteins and domains thereof. Other embodiments disclosed herein can include one or more constructs having a non-naturally occurring polypeptide or polynucleotide. In yet other embodiments, constructs disclosed herein can have a formula: (X—B)ₙ—Z, where X is at least one polypeptide encoding at least one domain of a first target protein or enzyme complex; Z is at least one polypeptide encoding at least one domain of a second target protein or enzyme complex; B is a polypeptide capable of linking X and/or Z or multiple domains of X and/or Z; and n is from 1 to 100. In certain embodiments, n can be 1, 2, 3, 4, 5, 6, 7, 8, 9, 10, 11, 12, 13, 14, 15, 16, 17, 18, 19, and/or 20 or up to 100. In accordance with these embodiments, the first or the second target protein can be the same or different target protein(s). In other embodiments, X and Z can be the same or different domain(s) of the first or the second target protein. In accordance of these embodiments, an in vivo library can include, but is not limited to, more than 10, 100, 1000, or 10,000 non-naturally occurring polypeptides having the formula: (X—B)ₙ—Z. In other embodiments, these non-naturally occurring polypeptide libraries can contain barcoded members for tracing the polypeptides of interest.

In certain embodiments, a construct contemplated herein can include one or more polypeptides that encode linker domains of one or more target polypeptides. In accordance with these embodiments, linker domains can include but are not limited to, Acyl Carrier Protein-Condensation Domain linkers (ACP Condensation), Acyl Carrier Protein-Heterocyclization Domain linkers (ACP Heterocyclization), Acyl Carrier Protein-Ketosynthase Domain linkers (AC-PKS), Acyl Carrier Protein-Thioesterase Domain linkers (ACP-TE), Adenylation Domain-Peptide Carrier Protein linkers (A-PCP), Acyltransferase Domain-Acyl Carrier Protein linkers (AT-ACP), Acyltransferase Domain-Dehydratase Domain linkers (AT-DH), Acyltransferase Domain-Ketoreductase Domain linkers (AT-KR), Condensation Domain-Adenylation Domain linkers (Condensation A), Dehydratase Domain-Enoylreductase Domain linkers (DH-ER), Dehydratase Domain-Ketoreductase Domain linkers (DH-KR), Dual Condensation/Epimerization Domain-Adenylation Domain linkers (Dual Condensation A), Enoylreductase Domain-Ketoreductase Domain linkers (ER-KR), Heterocyclization Domain-Adenylation Domain linkers (Heterocyclization A), (Both Acyl and Peptide) Carrier Protein-Condensation Domain linkers (Joint AC-PC), Ketoreducatse Domain-Acyl Carrier Protein linkers (KR-ACP), and Ketosynthase Domain-Acyltransferase Domain linkers (KS-AT). In certain exemplary embodiments, B of the formula, (X—B)ₙ—Z, can include one or more of these linker domains. In certain embodiments, domain linker can be about 10 to about 500 amino acids long. In other embodiments, domain linker sequences can be about 10 to about 450 amino acids long. In yet other embodiments, domain linker sequences can be categorized by a linker type.

In some embodiments, an exemplary construct can include at least 70, 75, 80, 85, 90, and/or 95 percent identity to at least one of the sequences referenced as SEQ ID NOs: 65-82, 108-143. In certain embodiments, an exemplary construct can include at least one sequence or fragment of a sequence represented by SEQ ID NOs: 108-143, for generating target molecules. In certain embodiments, constructs can be generated using two or more domains of an exemplary polypeptide, protein or enzyme. In accordance with these embodiments, the two or more domains of a target polypeptide, protein or enzyme can be modular megasynthases, polyketide synthases and/or non-ribosomal peptide synthases or hybrid molecules thereof. In certain embodiments, two or more domains of an exemplary target protein or enzyme can be two or more catalytic domains of an exemplary target protein or enzyme. In some embodiments, an exemplary construct can include at least 70, 75, 80, 85, 90, and/or 95 percent identity to at least one sequence represented by SEQ ID NOs: 33-64, or 108-143. In certain embodiments, an exemplary construct can include at least one of the sequences represented by SEQ ID NOs: 33-64, 109, 111, 113, 115, 117, 119, 121, 123, 125, 127, 129, 131, 133, 135, 137, 139, 141, 143 for generating target molecules.

In other embodiments, a construct contemplated herein can include one or more polypeptides that encode catalytic domains of one or more target polypeptide, protein or enzyme. In accordance with these embodiments, catalytic domains can include, but are not limited to, Acyltransferase (AT), Acyl Carrier Protein (ACP), Keto-Synthase (KS), Ketoreductase (KR), Dehydratase (DH), Enoylreductase (ER), Methyltransferase (MT), Sulfhydrolase (SH), and/or Thioesterase (TE). In certain exemplary embodiments, X and Z of the formula, (X—B)ₙ—Z, can include one or more of these catalytic domains. In other embodiments, X and Z can be the same or different domain(s) of the first or the second target polypeptide, protein or enzyme.

In certain embodiments, constructs generated herein are capable of synthesizing a secondary metabolite in a host (e.g. organism, microorganism or cell). The secondary metabolite can include, but are not limited to,

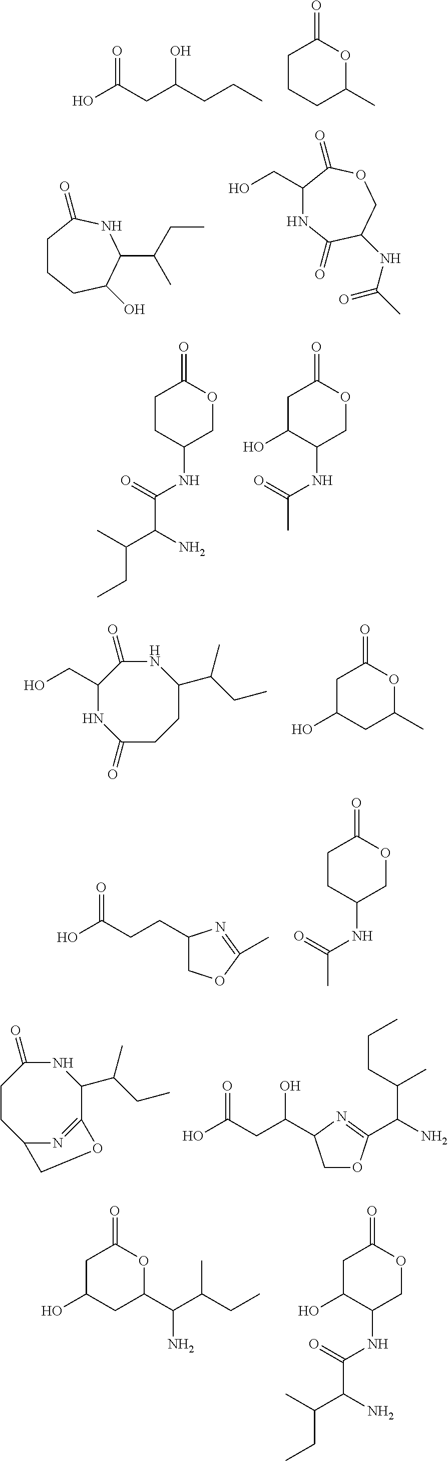

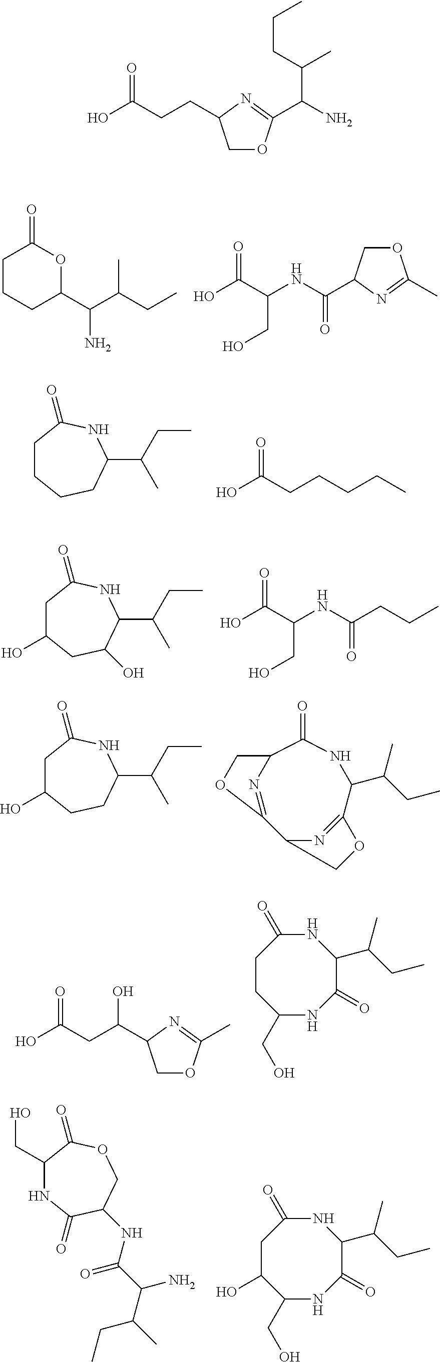

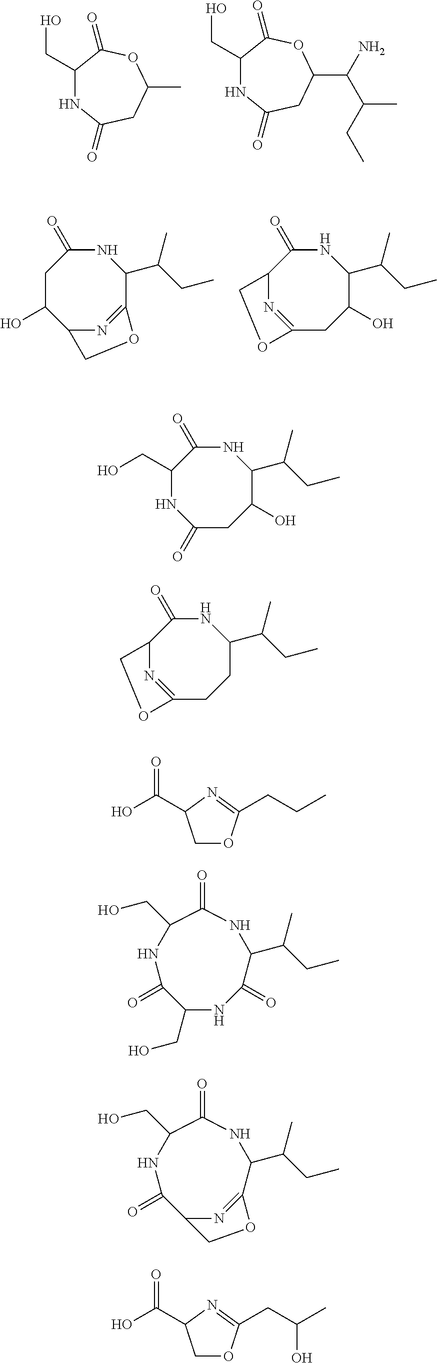

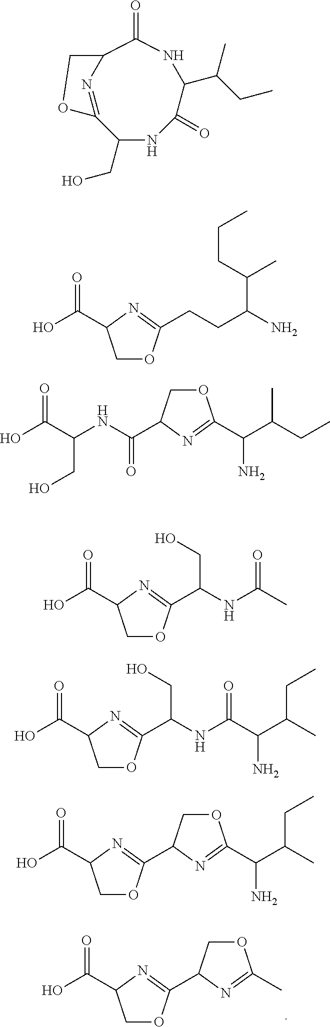

In some embodiments, constructs generated herein are capable of synthesizing a secondary metabolite, wherein secondary metabolites can include organic compounds not directly involved in normal growth, development, or reproduction of an organism (e.g. host organism). In certain embodiments, secondary metabolites can include natural or non-natural products or natural or non-natural molecules with chemical (e.g. fine chemical), pharmaceutical, flavor, or fragrance applications. In certain embodiments, secondary metabolites can include target molecules of polyketides, non-ribosomal peptides, and/or polyketide-non ribosomal peptide hybrids. In other embodiments, a secondary metabolite can include delta-hexalactone. In yet other embodiments, a secondary metabolite can include Rapamycin. In still other embodiments, a secondary metabolite can include Actinorhodin. In other embodiments, secondary metabolite can include Erythromycin A. In yet other embodiments, a secondary metabolite can include 6-Methylsalicylic acid. In certain embodiments, a secondary metabolite can include Aflatoxin B1. In further embodiments, a secondary metabolite can include Rifamycin S. In some embodiments, a secondary metabolite can include Lovastatin. In other embodiments, a secondary metabolite can include Amphotericin B. In other embodiments, a secondary metabolite can include Monensin A.

Some embodiments herein concern constructs for compiling an in vivo library of one or more target molecules for synthesis in a microorganism. In accordance with these embodiments, constructs can be generated that encompass one or more genetic variation(s) of a gene or gene segment corresponding to a target catalytic domain of a polypeptide or protein (e.g. enzyme). In certain embodiments, the construct can include a barcode or a tag for trackability. In other embodiments, the barcode can be positioned outside of the open reading frame of the gene or gene segment. It is contemplated that these comprehensive libraries can be generated for any eukaryotic or prokaryotic polypeptide, protein, trait or pathway, chemical or small molecule. In certain embodiments, engineered cells or organisms (e.g. microorganisms) can be used to produce constructs contemplated herein.

Certain embodiments can include a non-naturally occurring polynucleotide encoding a construct having the formula: (X—B)ₙ—Z, as disclosed above. In accordance with these embodiments, the first or the second target protein can be the same or different target protein(s). In other embodiments, X and Z can be the same or different domain(s) of the first or the second target protein. In accordance with these embodiments, X and Z can be the same or different catalytic domain(s) of a megasynthase. In accordance with these embodiments, B can be a linker selected from naturally-occurring or non-naturally occurring linkers of megasynthase catalytic domains that when assembled with X and Z form a non-naturally occurring megasynthase construct capable of creating novel constructs for producing target agents in a cell or organism. In certain embodiments, the polynucleotide encoding the construct having the formula: (X—B)ₙ—Z can be created by codon optimization or codon harmonization.

In some embodiments, polynucleotides disclosed herein include, but are not limited to, a traceable barcode positioned outside of the gene or the gene segment open reading frame, wherein the traceable barcode corresponds to or is quantitatively linked to a genetic variation of the gene or the gene segment. In accordance of these embodiments, an in vivo library or trackable library can include, but is not limited to, more than 10, 100, 1000, or 10,000 non-naturally occurring polynucleotides encoding the construct having the formula: (X—B)ₙ—Z. In accordance with these embodiments, a trackable library can include a barcoded library.

In other embodiments, an exemplary polynucleotide encoding the construct having the formula: (X—B)ₙ—Z can include at least 70, 75, 80, 85, 90, and/or 95 percent identity to at least one of the sequences referenced as SEQ ID NOs: 1-32, 108, 110, 112, 114, 116, 118, 120, 122, 124, 126, 128, 130, 132, 134, 136, 138, 140, 142. In certain embodiments, an exemplary (X—B)ₙ—Z construct can include at least one of the sequences represented by SEQ ID NOs: 1-32, 108, 110, 112, 114, 116, 118, 120, 122, 124, 126, 128, 130, 132, 134, 136, 138, 140, 142 for generating target molecules.

Other embodiments herein concern methods for generating a construct of the formula, (X—B)ₙ—Z, disclosed herein. In accordance with these embodiments, a method can include obtaining at least one polypeptide sequence encoding at least one domain (e.g. catalytic) of one or more target proteins; determining a linker sequence capable of linking the at least one polypeptide encoding at least one domain of the one or more target protein to the linker; and generating a construct having the at least one polypeptide sequence on either side of a linker sequence. In other embodiments, determining a linker sequence further includes creating a gene cluster annotation of a target gene, and/or converting a construct having an amino acid sequence into at least one nucleotide sequence using codon harmonization.

Certain embodiments herein concern assessing and scoring genetic variations of genes or gene segments of one or more target proteins that affect one or more residue of the target protein(s). In accordance with these embodiments, constructs can be traced to one or more variation positively affecting protein function and that contribute to an overall trait. In accordance with these embodiments, these variations can be selected for and used for creating modulated engineered biologics, biopharma products, cells, or organisms having or producing a construct disclosed herein.

Yet other embodiments disclosed herein provide methods for generating a target molecule using a CRISPR enabled trackable genome engineering (CREATE) vector having an editing cassette and/or at least one guide RNA (gRNA). The editing cassette can include a region which is homologous to a target region of a nucleic acid in the cell, a mutation of at least one nucleotide relative to the target region, and/or a protospacer adjacent motif (PAM) mutation. In some embodiments, the CREATE editing cassette introduces a silent PAM mutation that protects from CRISPR cutting, coupled to the target mutation. The gRNA can include a region complementary to a portion of the target region and/or a region that recruits a Cas9 nuclease. A CREATE vector can be used to make a targeted and trackable genomic mutation. In some embodiments, CREATE can be used to change the ‘chassis’ substrate specificity, altering AT and A domain specificities, expanding the biosynthesis library from 32 to >10000 members.

In certain embodiments, an organism can be a eukaryotic cell or a microorganism (e.g. bacteria, yeast, fungus, or other microorganism) capable of being genomically-engineered or manipulated, for example, for improved synthesis or production of a byproduct of the organism or synthesis or production of a novel molecule. In other embodiments, compositions and methods disclosed herein are directed at producing genomically-engineered eukaryotic or prokaryotic cells, for example, cancer cells, product-producing cells (e.g., insulin, growth factors, and other biologics), tissue cells and others known in the art. In yet other embodiments, compositions and methods disclosed herein are directed at producing genomically-engineered microorganisms, for example, bacteria (e.g., E. coli). In accordance with these embodiments, bacteria can be engineered to house a construct (e.g. a construct of the formula, (X—B)ₙ—Z) disclosed herein in order to product target agents.

Trackable agents contemplated of use in any of the disclosed compositions or methods can include, but are not limited to barcodes. In accordance with these embodiments, barcodes can be, but are not limited to, DNA sequences (e.g., 20-1,000 nucleotides in length) or other agents known by those skilled in the art. Because barcodes can be physically linked to a specific allele cassette they can be used to track the presence of each synthetic oligo as well as track each engineered cell or microorganism within a mixed population. In other embodiments, barcodes can be further selected to exclude sequences that would lead to cleavage of DNA during library synthesis and sequences that contain more than six bases identical to the regions used to amplify the tag sequences.

Some embodiments disclosed herein can include modifying microorganisms or cells to express one or more construct (e.g. conserved or mutated domain). In accordance with these embodiments, a mutated domain can be a mutated catalytic domain originating from a catalytic domain of a megasynthase. These manipulated cells or microorganisms can then be selected to produce known or novel target agents such as small molecules, biopharma agents, biofuels, fusion molecules, recombinants or biologics.

### DESCRIPTION OF ILLUSTRATIVE EMBODIMENTS

### Definitions

As disclosed herein “modulate” can mean an increase, a decrease, upregulation, downregulation, an induction, a change in encoded activity, a change in stability or the like, of one or more of targeted genes or gene clusters.

As disclosed herein “module” can mean a specific sequence of DNA designed to have a specific effect when introduced to a cell. The effect could be to target the module to a specific part of the genome or to a specific cellular location, to result, in for example, a modulation as defined above, or to enable easier quantification via genomics technologies among others.

As disclosed herein “measurement of biological effect” can be a comparison of one cellular trait resulting from one genetic variation with respect to another cellular trait resulting from a second genetic variation or compared to a control with no variation. Examples of measurement of biological effect include, but are not limited to, comparison of the rate of growth of two cell types, comparison of the color of two cell types, comparison of the fluorescence of two cell types, comparison of a metabolite concentration within two cell types, comparison of lag phase of two cells types, comparison of the survival of two cell types, comparison of the consumption of a an agent by two cell types, comparison of production rates of an agent of two cell types, comparison of two or more mutations on a target protein, analysis of effects of a protein activity due to genetic variation and other parameters.

As disclosed herein, a secondary metabolite can mean an organic compound that is not directly involved in the normal growth, development, or reproduction of an organism.

As disclosed herein “genetic modification” or “genetic variation” can mean any change(s) to a composition or structure of DNA (whole genes or gene segments) with respect to its function within an organism. Genetic modification examples include, but are not limited to, deletion of nucleotides from cell, insertion of nucleotides to cell, rearrangement of nucleotides or changes that create an amino acid change in a protein coded form by the DNA.

As disclosed herein “multiplex modification” can mean creating two or more genetic modifications in the same experiment. These modifications can occur within the same cell or within separate cells.

As disclosed herein “tracking” can mean any nucleotide sequence that can be used to identify or trace a genetic modification, directly or indirectly. Examples of tracking include, but are not limited to, nucleotide sequences that can be identified by sequencing technologies, nucleotide sequences that can be identified by hybridization technologies, nucleotide sequences that create a bioproduct that can be identified, such as a protein identified by proteomic technologies or molecule identified by common analytical techniques (e.g., chromatography and/or spectroscopy).

As disclosed herein “functional module” can mean any nucleotide sequence inserted, rearranged, and/or removed at genetic locus (loci). A functional module elicits primary effect(s) on gene loci (locus) that can be predicted or anticipated. Functional module examples and corresponding primary effects include, but are not limited to, insertion of a promoter that cause a change of RNA transcription, alteration of nucleotides involved in translation initiation, deletion of nucleotides that make up part/all of the reading frame of a gene resulting in loss of gene product, insertion of sequence that causes a change in gene product, and deletion of sequence that interacts with a small molecule that causes an effect to be less dependent on the small molecule.

As disclosed herein “vector” can be any of a variety of nucleic acids that include a sought-after or target sequence or sequences to be delivered to or expressed in a cell or organism. The sought-after sequence(s) can be included in a vector, such as by restriction and ligation or by recombination. Vectors can typically be composed of DNA, although RNA vectors are also available. Vectors include, but are not limited to: plasmids, fosmids, phagemids, virus genomes and artificial chromosomes.

### BRIEF DESCRIPTION OF THE FIGURES

The following drawings form part of the present specification and can be included to further demonstrate certain embodiments of the present disclosure. The embodiments can be better understood by reference to one or more of these drawings in combination with the detailed description of specific embodiments presented herein.

FIG. 1 represents a model of clustering analysis for various linkage classes of use in embodiments described herein (provided in color upon request).

FIG. 2 is a schematic diagram representing a method for generating a construct of one embodiment described herein.

FIG. 3 is a schematic diagram representing loading modules, extension modules, and the naming scheme of an exemplary construct.

FIG. 4 represents schematic diagrams illustrating: computational mining and linker design; and gene design and assembly of some embodiments described herein.

FIG. 5A represents a schematic diagram illustrating exemplary computational mining for potential linker sequences of use in constructs contemplated herein, as used in methods disclosed herein.

FIG. 5B represents a schematic diagram illustrating a design of a target linker, as described in exemplary embodiments herein.

FIG. 6A illustrates an exemplary targeted design of a construct (e.g. modular megasynthase), of one embodiment of the instant disclosure.

FIG. 6B illustrates a linker region from computational mining compared to a linker from known structures of some embodiments described herein.

FIG. 6C illustrates a linker region from computational mining compared to a linker from known structures of some embodiments described herein as derived from FIG. 6B.

FIG. 7A-7D represents an exemplary process illustrating: 7A) codon harmonization and synthesis of fragments by methods known in the art as used in exemplary embodiments herein; 7B) yeast cloning of exemplary fragments; 7C) second step yeast cloning of exemplary fragments and 7D) integration of exemplary fragments into a genome of a microorganism of some embodiments described herein.

FIG. 8A-8C illustrate: 8A) the mass spectrum of a target metabolite as compared to a control; and 8B-8C: the mass spectrum of a target molecule against a standard of the same molecule to demonstrate synthesis using some exemplary methods described herein.

FIG. 9 illustrates target molecules produced by an exemplary construct expressed in an exemplary microorganism using embodiments disclosed herein.

FIG. 10 illustrates one strategy for combinatorial assembly of barcoded exemplary enzymes (e.g. modular megasynthases) of certain embodiments disclosed herein.

FIG. 11A-11B illustrates a method for generating a mutated enzyme construct of certain embodiments disclosed herein using gene editing of certain embodiments disclosed herein.

FIG. 12A illustrates a block diagram for certain exemplary linker sequences in color-coded blocks (provided in color upon request) of certain embodiments disclosed herein.

FIG. 12B illustrates an exemplary ketosynthase-acyltransferase linker sequence of use in certain constructs of various embodiments disclosed herein.

FIG. 13A represents schematic diagrams illustrating CRISPR Enabled Trackable Genome Engineering (CREATE) cassette and design of use for constructs of certain embodiments disclosed herein.

FIG. 13B illustrates Protospacer Adjacent Motif (PAM) mutation and editing introduced to certain constructs (e.g. a catalytic domain) of constructs of certain embodiments disclosed herein.

FIG. 13C illustrates a CREATE strategy of use for certain embodiments disclosed herein.

FIG. 14A illustrates an exemplary method using in vivo gene editing referred to as CREATE of use for certain embodiments disclosed herein.

FIG. 14B illustrates an exemplary mutation generated in an exemplary target molecule using CREATE for certain embodiments disclosed herein.

FIG. 15 illustrates use of a barcoded-Tracking Combinatorial Engineering (bTRACE) system of some embodiments described herein.

### DETAILED DESCRIPTION

In the following sections, various exemplary compositions and methods are described in order to detail various embodiments of the disclosure. It will be obvious to one skilled in the art that practicing the various embodiments does not require the employment of all or even some of the details outlined herein, but rather that concentrations, times, temperature and other details can be modified through routine experimentation. In some embodiments, well known or previously disclosed methods or components have not been included in the description.

In accordance with embodiments of the present invention, there can be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986) as well as other techniques known in the art applicable to embodiments disclosed herein.

Certain embodiments disclosed herein concern compositions, systems, methods, and uses for generating comprehensive in vivo libraries related to genetic variations for producing target molecules such as proteins, peptides, polypeptides, target agents, small molecules and chemicals. In certain embodiments, target molecules can be prokaryotic or eukaryotic target polypeptides, peptides, proteins or other agents of use in a variety of applications. In other embodiments, target molecules can be generated related to producing biofuels, biotech agents and biopharmaceutical agents or chemicals of use for small or large scale production or screening using combinatorial enzyme biosynthesis systems disclosed herein.

To date, combinatorial enzyme biosynthesis efforts have been limited by a lack of tools for designing, building, and testing target complex enzymes of use in production of target molecules. For example, megasynthases are complex multienzyme complexes. These multienzyme complexes are protein complexes having multiple catalytic domains connected together with structured linker regions in a single polypeptide chain to permit functionality of the catalytic domains. Megasynthases are the foundation of many biological processes and perform a vast array of biological functions. Linker regions of megasynthases confer the requisite structure for constructive interactions between catalytic domains and groups of catalytic domains (modules) in order to perform a variety of tasks within an organism. In part, lack of attention to these linker regions has been implicated as one issue in the limited successes of previous studies using these complex enzymes in combinatorial biosynthesis. For example, linker sequences of megasynthases as well as their combination with particular catalytic domains are critical for proper function or manipulation to form “modular” megasynthases (a non-naturally occurring megasynthase) capable of producing target molecules of constructs disclosed herein. Megasynthases are fairly simple regarding their hierarchical and modular architecture, but have not been as easily re-programmable as anticipated due-in part-to their complex structure and dynamics. As such, advances in synthetic biology have not, until the instant disclosure, been previously implemented as a framework for the construction of “modular” megasynthases which begins with designing a set of all potentially required parts (e.g. catalytic domains and corresponding linker regions) and the hierarchical assembly of these parts into a variety of “modular” megasynthases (hereinafter “modular megasynthases”). Embodiments herein provide for a highly efficient, scalable platform approach to creating modular megasynthase design and assembly for combinatorial biosynthesis as scaffold for production of a multitude of target molecules.

Embodiments herein provide for a platform technology that can be used to design a set of context-independent parts that behave predictably, regardless of the broader enzyme design, enabling simple, scalable, and combinatorial assembly of multienzyme complexes, such as reprogrammable modular megasynthases of use to produce target molecules. In certain embodiments, this platform can include a computational design pipeline for context-independent linker sequences that, when combined with the predetermined catalytic domains (e.g. of a modular megasynthase) using various techniques known in the art, can be assembled leading to a system for producing novel molecules in a microorganism or a cell.

In certain embodiments, compositions, methods and used for creating “reconfigured” modular megasynthase constructs (i.e. non-naturally occurring modular megasynthases) for combinatorial biosynthesis for diverse generation of these constructs through the use of genome engineering tools are disclosed. As disclosed herein, diversity can be generated at different levels of construction of the hierarchical architecture of these synthetic or unnatural modular megasynthases. In accordance with these embodiments, diversity can be generated in order or alignment of modules (e.g. domains) within a gene; selection based on function of the modules; and substrate specificity of selected modules. In certain embodiments, using design of modules (catalytic domains and linker domains) with varied function and mixing and matching of target modules diversity of the system can be generated. In other embodiments, diversity can be generated through methods disclosed herein such as alteration of the module substrate specificity through in vivo genome engineering. Yet other embodiments include methods for creating mutations within the modules of use to further diversify target molecules produced by constructs described herein. One advantage of this approach is creating methods that do not require the cloning of a targeted construct for synthesis of the target molecules. In certain embodiments, methods disclosed herein can use CREATE, a CRISPR-based technology for synthesizing constructs which contain an editing cassette and CRISPR-RNA sequentially for example, of use for creating mutants and other constructs.

Understanding relationships between a protein's amino acid structure and function is critical in protein engineering efforts, which are increasingly commonplace in almost all drug development programs (e.g., whether focused on protein-based therapies or enzyme driven synthesis of pharmaceutical products). In certain embodiments, protein design criteria grow increasingly stringent, including efforts to simultaneously alter multiple characteristics of a target protein such as stability, catalytic activity, target specificity, pharmacokinetic activity, shelf-life, among others depending on the application.

Megasynthases are composed of sets of domains that sequentially catalyze various reactions, ultimately leading to compounds that are non-essential to growth, development, or reproduction of a host organism (e.g., secondary metabolites). Sub-classes of megasynthases can include, but are not limited to, polyketide synthases (PKSs) and non-ribosomal peptide synthases (NRPSs).

Polyketide synthases (PKSs) are a family of multifunctional enzymes that assemble the core structures of polyketides via the sequential Claisen-like condensations of extender units derived from carboxylated acyl-CoA precursors in a linear fashion. At minimum, each module of a PKS contains covalently-linked ketosynthase (KS), acyltransferase (AT), and acyl-carrier protein (ACP) domains. There are also optional tailoring domains such as ketoreductase (KR), dehydratase (DH), and enoylreductase (ER) domains that incorporate different functionality into the polyketide. In addition, there is a distinct logic to the organization of catalytic domains within each PKS module, beginning with a KS, followed by an AT, the three optional tailoring domains, and finally, the ACP.

In some embodiments, synthesis of a PKS begins on the loading module with an AT loading an acyl-CoA derivative onto an ACP. In the next module (the first extension module), a KS then condenses the acyl-CoA derivative on the loading ACP with the acyl-CoA derivative on the next ACP down the chain, generating a ketide. The carbonyl in this ketide then undergoes various reductions, depending on the reductive tailoring domains present in the module. For example, a KR reduces the ketide to an alcohol. A KR and DH reduces the ketide to an alcohol and then reduces the alcohol to an alkene. A KR, DH, and ER produces a fully reduced hydrocarbon. The ketide produced by a PKS is dependent on the number of modules in the enzyme and the domain structure of these modules. Finally, a thioesterase (TE) hydrolyzes the ketide from the enzyme, preferably intramolecularly using an alcohol, generating a lactone or, with water, generating an organic acid. Exemplary PKS include but are not limited to Ac-Mal_{H}-Mal_{OH}, Ac-Mal_{OH}-Mal_{OH}, Ac-Mal_{H}-Mal_{OH}, and Ac-Mal_{OH}-Mal_{H}.

Based on the structures of the polyketide products, as well as biochemical features of the PKSs, PKSs are currently classified into types I, II, and III subgroups. Type I PKSs are megasynthases in which catalytic domains are typically found in a single polypeptide. A modular type I PKS, such as the 6-deoxyerythronolide B synthase (DEBS), consists of multiple modules and each module catalyzes one round of chain elongation and modification. Linear juxtaposition of modules facilitates unidirectional transfer of the growing polyketide from the upstream to the downstream modules in assembly line-like fashion. Type II PKSs are involved in the synthesis of aromatic polyketides, such as the aglycons of actinorhodin. Type III PKSs, such as chalcone synthase, are homodimeric PKSs that synthesize smaller aromatic compounds in bacteria, fungi, and plants. The linear arrangement of domains and modules provides a general guidance to reprogram these highly modular megasynthases.

Polyketides, synthesized by PKSs, are found in soil-borne or marine actinomycetes bacteria, filamentous fungi, and plants. Unfortunately, many of these organisms are difficult to work with in both laboratory and industrial settings. For example, the original strains are generally difficult to culture (long doubling times) or domesticate and they are genetically intractable and refractory toward common molecular biology tools. Moreover, the polyketide biosynthetic pathways are weakly expressed or silent under laboratory culturing conditions, resulting in low polyketide titers. Therefore, other microorganisms were investigated to be of use to introduce these complexes to in effort to create a system for generating a variety of target molecules using modified multiplex enzyme constructs.

Nonribosomal peptide synthases (NRPSs) are another class of enzyme that have similar modularity, hierarchical architecture, and logic to PKSs. The main difference lies in their synthesis, in that instead of acyl-CoA derivatives, NRPSs use adenylated amino acids as their substrates. Using amino acids dictates that NRPSs contain different catalytic domains within modules, and they have an adenylation domain (A) that adenylates and loads an amino acid onto a peptide carrier protein (PCP) and a condensation domain (C) that condenses amino acids from two PCPs. An exemplary NRPS is Ile-Ser-Ser.

There is also a class of modular megasynthases termed PKS-NRPS hybrids, as they contain modules of both PKS and NRPSs. Exemplary PKS-NRPS hybrids include but are not limited to Ac-Ser-Mal_{OH}, Ac-Ser-Mal_{H}, Ile-Mal_{OH}-Mal_{OH}, Ile-Mal_{OH}-Mal_{H}, Ile-Mal_{OH}-Mal_{OH}, Ile-Mal_{H}-Mal_{OH}, Ac-Mal_{OH}-Ser, Ac-Mal_{H}-Ser, Ile-Ser-Mal_{OH}, Ile-Ser-Mal_{H}, Ac-Ser-Ser, Ile-Mal_{OH}-Ser, Ile-Mal_{H}-Ser.

In some embodiments, microorganisms such as bacteria, a non-natural host can be used as a host for modified modular megasynthases. In accordance with these embodiments, Escherichia coli (E. coli) can be used for the reconstitution, manipulation, and optimization of domains/linkers of a megasynthase to construct such as system for producing a diverse variety or related and unrelated target molecules at a micro or macro scale. For example, Escherichia coli (E. coli) can be used for reconstitution, manipulation, and optimization of polyketide biosynthesis in part due to: (1) ease of culturing and fast growth characteristics; (2) availability of superior genetic tools; (3) well-understood primary metabolism; and (4) lack of endogenous polyketide pathways that may crosstalk or interfere with transplanted pathways.

In other embodiments, an organism of use to house modular complexes of the present disclosure can be a eukaryotic cell, bacteria, yeast, fungi, or other microorganism capable of being genomically-engineered or manipulated, for example, for improved synthesis or production of a natural or non-natural byproduct (e.g. secondary metabolites) of the organism.

It is understood by those of skill in the art that polyketide biosynthesis revealed that its corresponding genes are highly modular, producing megasynthases that perform much like an assembly line. Multiplex enzymes, such as, megasynthases can include, but are not limited to, polyketide synthases (PKSs) and non-ribosomal peptide synthases (NRPSs). The ability to reprogram these highly modular megasynthases has provided molecular biologists access to the biological activity and structural diversity of natural products. It has been demonstrated that small molecule diversity can be produced by manipulating PKS genes in three ways: 1) by adding or removing entire extension modules from the PKS, influencing the size of the small molecule (scaffold length); 2) by altering the reduction domains to completely reduce, partially reduce, or not reduce each acyl unit, influencing the functional groups present on the small molecule (scaffold structure); and 3) by altering the specificities of the acyl transferases that load each module, influencing the structure and functionality of the small molecule (scaffold specificity). However, due to the size and complexity, multiplex enzymes having various combinations of modules were extremely difficult to express in tested E. coli. Further, absence of sufficient techniques including in vivo mutagenesis to manipulate and alter these multiplex enzymes limited generation of a full-scale combinatorial library.

Methods described herein include computationally designing microbial biosynthetic machinery such as polyketide synthases (PKSs) and non-ribosomal peptide synthases (NRPSs) specifically for microorganisms and other hosts (e.g. E. coli) and then refactoring them in massive multiplex. A computational tool that searches publically available bacterial genomes for design rules specific to these genes is described herein. Design rules output by this program are then used to build synthetic genes that produce compounds of interest.

Other embodiments include methods for designing a non-naturally occurring PKS construct by shuffling or combining catalytic domains of PKS into a certain arrangement or combination of interest. The catalytic domains that can be used to create a non-naturally occurring PKS include, but are not limited to, Acyltransferase (AT), Acyl Carrier Protein (ACP), Keto-Synthase (KS), Ketoreductase (KR), Dehydratase (DH), Enoylreductase (ER), Methyltransferase (MT), Sulfhydrolase (SH), and/or Thioesterase (TE). In exemplary embodiments, two or more of these domains are linked together to create a non-naturally occurring PKS construct. In certain embodiments, the exemplary construct is capable of synthesizing a secondary metabolite in an organism. Some embodiments concern methods for creating an appropriate linker sequence that is capable of linking two or more of these catalytic domains. The linker sequence can be a polypeptide or a polynucleotide that is capable of maintaining the structure and function of a target protein or a target gene, respectively.

Some embodiments herein concern constructs for compiling an in vivo library of one or more target proteins. Certain embodiments can include a construct having a non-naturally occurring polypeptide or polynucleotide. Other embodiments can include a construct having the formula: (X—B)ₙ—Z, where X is at least one polypeptide encoding at least one domain of a first target protein; Z is at least one polypeptide encoding at least one domain of a second target protein; B is a polypeptide capable of linking X and/or Z; and n is 1 to 100. In accordance with these embodiments, the first or the second target protein can be the same or different target protein(s). In other embodiments, X and Z can be the same or different domain(s) of the first or the second target protein. In some embodiments, n can be 1, 2, 3, 4, 5, 6, 7, 8, 9, 10, 11, 12, 13, 14, 15, 16, 17, 18, 19, and/or 20 or more.

In other embodiments, an exemplary construct can include at least 70, 71, 72, 73, 74, 75, 76, 77, 78, 79, 80, 81, 82, 83, 84, 85, 86, 87, 88, 89, 90, 91, 92, 93, 94, 95, 96, 97, 98, and/or 99 percent identity to at least one of SEQ ID NOs: 33-64, 109, 111, 113, 115, 117, 119, 121, 123, 125, 127, 129, 131, 133, 135, 137, 139, 141, 143.

In other embodiments, an exemplary linker can include at least 70, 71, 72, 73, 74, 75, 76, 77, 78, 79, 80, 81, 82, 83, 84, 85, 86, 87, 88, 89, 90, 91, 92, 93, 94, 95, 96, 97, 98, and/or 99 percent identity to at least one of the polypeptide of SEQ ID NOs: 70-107.

In other embodiments, an exemplary polynucleotide encoding a linker can include at least 70, 71, 72, 73, 74, 75, 76, 77, 78, 79, 80, 81, 82, 83, 84, 85, 86, 87, 88, 89, 90, 91, 92, 93, 94, 95, 96, 97, 98, and/or 99 percent identity to at least one of the polynucleotide of SEQ ID NOs: 72, 75, 80, 82, 86, 91, 96, and 99.

In other embodiments, an exemplary KS-AT linker can include at least 70, 71, 72, 73, 74, 75, 76, 77, 78, 79, 80, 81, 82, 83, 84, 85, 86, 87, 88, 89, 90, 91, 92, 93, 94, 95, 96, 97, 98, and/or 99 percent identity to at least one of the Conserved Motif polypeptide of SEQ ID NOs: 103-107.

In other embodiments, the constructs disclosed herein can include a polypeptide construct that encodes catalytic domains of one or more target molecules such as polypeptides. In accordance with these embodiments, catalytic domains can include, but are not limited to, Acyltransferase (AT), Acyl Carrier Protein (ACP), Keto-Synthase (KS), Ketoreductase (KR), Dehydratase (DH), Enoylreductase (ER), Methyltransferase (MT), Sulfhydrolase (SH), and/or Thioesterase (TE). In certain exemplary embodiments, X and Z of the formula, (X—B)ₙ—Z, can include these named domains or other similar domains known in the art or to be discovered. In other embodiments, X and Z can be the same or different domain(s) of the first or the second target protein. For example, an exemplary construct can include a polypeptide having the formula, (X—B)ₙ—Z, where X includes Acyltransferase (AT), Z includes Acyl Carrier Protein (ACP), and B is a polypeptide capable of linking X and Z. Yet other embodiment, X can include, but is not limited to, Acyltransferase (AT), Acyl Carrier Protein (ACP), Keto-Synthase (KS), Ketoreductase (KR), Dehydratase (DH), Enoylreductase (ER), Methyltransferase (MT), Sulfhydrolase (SH), and/or Thioesterase (TE), and Z can include, but is not limited to, Acyltransferase (AT), Acyl Carrier Protein (ACP), Keto-Synthase (KS), Ketoreductase (KR), Dehydratase (DH), Enoylreductase (ER), Methyltransferase (MT), Sulfhydrolase (SH), and/or Thioesterase (TE). In other embodiments, X and Z can be the same or different domain(s) of the first or the second target protein.

In certain embodiments, constructs disclosed herein are capable of synthesizing a secondary metabolite or non-naturally occurring target molecule in a manipulated organism housing a modified system of modules disclosed herein. Exemplary secondary metabolites can include, but are not limited to, antibiotics or derivatives thereof, biologics, pharma agents and the like. In certain embodiments, secondary metabolites can include, but are not limited to, Rapamycin, Actinorhodin, Erythromycin A, 6-Methylsalicylic acid, Aflatoxin B1, Rifamycin S, Lovastatin, Amphotericin B, and Monensin A and other molecules. In certain embodiments, secondary metabolites include natural or non-natural products or molecules with fine chemical, pharmaceutical, flavor, or fragrance applications. In certain embodiments, secondary metabolites include target molecules of polyketides, non-ribosomal peptides, and/or polyketide-non ribosomal peptide hybrids.

Certain embodiments include a non-naturally occurring polynucleotide encoding the construct having the formula: (X—B)ₙ—Z, where X is at least one polypeptide encoding at least one domain of a first target protein; Z is at least one polypeptide encoding at least one domain of a second target protein; B is a polypeptide capable of linking X and/or Z; and n is 1 to 100. In some embodiments, n is 1, 2, 3, 4, 5, 6, 7, 8, 9, 10, 11, 12, 13, 14, 15, 16, 17, 18, 19, and/or 20. In accordance with these embodiments, the first or the second target protein can be the same or different target protein(s). In other embodiments, X and Z can be the same or different domain(s) of the first or the second target protein. In certain embodiments, the polynucleotides can include, but are not limited to, a traceable barcode positioned outside of the gene or the gene segment open reading frame, wherein the traceable barcode corresponds to or is quantitatively linked to a genetic variation of the gene or the gene segment.

In other embodiments, an exemplary polynucleotide can include at least 70, 71, 72, 73, 74, 75, 76, 77, 78, 79, 80, 81, 82, 83, 84, 85, 86, 87, 88, 89, 90, 91, 92, 93, 94, 95, 96, 97, 98, and/or 99 percent identity to at least one of SEQ ID NOs: 1-32, 108, 110, 112, 114, 116, 118, 120, 122, 124, 126, 128, 130, 132, 134, 136, 138, 140, 142.

Other embodiments herein concern methods for generating a construct having a non-naturally occurring polypeptide or polynucleotide. Certain embodiments include methods of: obtaining at least one polypeptide sequence encoding at least one domain of one or more target proteins; determining a linker sequence that are capable of linking the at one or more polypeptide encoding at least one domain of the one or more target protein; and generating a construct having the at least one polypeptide sequence and the linker sequence. In other embodiments, the step of determining a linker sequence further includes creating a gene cluster annotation of the target gene, and/or converting the construct having an amino acid sequence into at least one nucleotide sequence by using codon harmonization in order to determine one or more linkers of use to create modules (domains with a linker) of a modular megasynthase.

Directed evolution can be a powerful engineering and discovery tool, but random and often combinatorial nature of mutations makes their individual impacts difficult to quantify and thus challenges further engineering. More systematic analysis of contributions of individual residues (e.g., saturation mutagenesis) remains labor- and time-intensive for entire proteins and simply is not possible on reasonable timescales for multiple proteins in parallel (metabolic pathways, multi-protein complexes) using standard methods.

Advances in multiplex oligonucleotide synthesis, recombineering, and DNA assembly are radically changing genetic engineering with broad implications across biology and biotechnology in general. This technology can be used to rapidly and efficiently examine the roles of all genes in a microbial or eukaryotic genome using mixtures of barcoded oligonucleotides. See for example U.S. Patent Publication No. 2015/0368639, disclosure of which is incorporated by reference in its entirety to the extent they are not inconsistent with the explicit teachings of this specification.

Genetic manipulation (e.g., using whole genes or gene fragments disclosed herein) of genes encoding a protein can be used to make desired genetic changes (e.g. mutations, insertions, deletions etc.) that can result in desired phenotypes and can be accomplished through numerous techniques including but not limited to, i) introduction of new genetic material, ii) genetic insertion, disruption or removal of existing genetic material, as well as, iii) mutation of genetic material (e.g., point mutations or cluster point mutations) or any combinations of i), ii), and iii), that results in desired genetic manipulations with desired phenotypic changes. Mutations can be directed (e.g., site-directed) or random, utilizing any techniques such as insertions, disruptions or removals, in addition to those including, but not limited to, error prone or directed mutagenesis through PCR, mutation strains, and random mutagenesis.

In some embodiments herein, disclosed methods demonstrate abilities for inserting and accumulating higher order modifications into a microorganism's genome or a target protein. These mutations are not confined only to sequences of regulatory modules, but can also extend to protein-coding regions. Protein coding modifications can include, but are not limited to, amino acid changes, codon optimization, codon harmonization, and translation tuning.

In one embodiment, methods can include a barcoded-Tracking Combinatorial Engineering (bTRACE). Briefly, bTRACE uses a persistent barcode sequencing and multiplexed binary assembly to enable tracking of mutations and quantification of mutations on a population wide level. For example, each member of the library is barcoded, and using multiplex linking PCR, various characteristics of each gene (e.g., module types and specificities) can be assembled to the barcode. These assembled constructs are MiSeq compatible. Once qualitative characteristics of the library are connected to barcodes, more quantitative data can be collected by sequencing just the HiSeq compatible barcodes. See for example also Zeitoun et al., Quantitative Tracking of Combinatorially Engineered Populations with Multiplexed Binary Assemblies, ACS SYNTH BIOL. 2017 Jan. 24; and International Patent Publication No. WO 2015/123339, disclosures of which are incorporated by reference in its entirety to the extent they are not inconsistent with the explicit teachings of this specification.

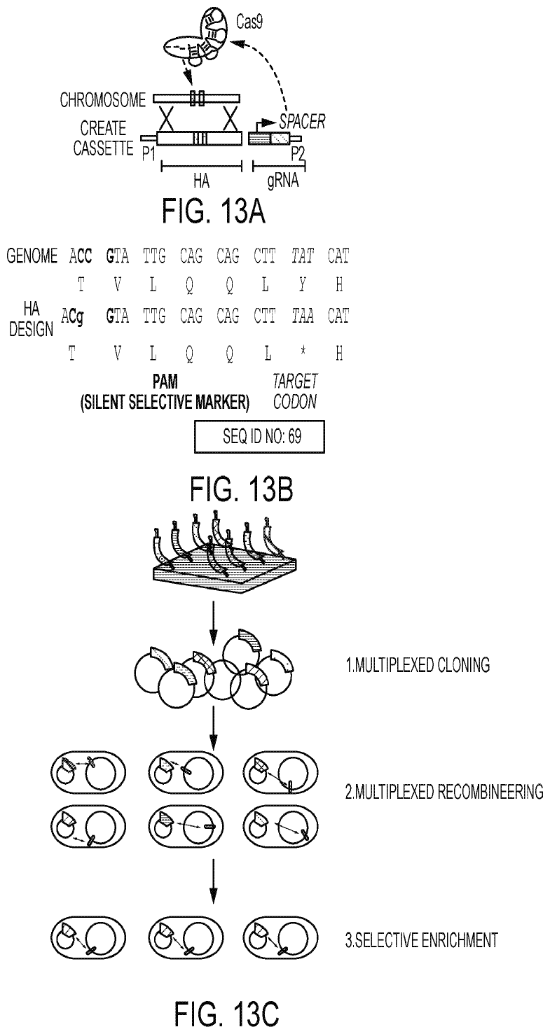

Also described herein are methods for trackable, precision genome editing using a CRISPR-mediated system referred to as CRISPR enabled trackable genome engineering (CREATE). Clustered regularly interspersed short palindromic repeats (CRISPR) exist in many bacterial genomes and have been found to play an important role in adaptive bacteria immunity. The CREATE methods achieve high efficiency editing/mutating using a single vector that encodes both an editing cassette and a guide RNA (gRNA)). CREATE editing cassette introduces a silent protospacer adjacent motif (PAM). The PAM mutation can be any insertion, deletion or substitution of one or more nucleotides that mutates the sequence of the PAM such that the mutated PAM (PAM mutation) is not recognized by the CRISPR system. For example, a cell that includes a PAM mutation can be said to be “immune” to CRISPR-mediated killing (see for example FIG. 13B) in part, due to this lack of recognition.

Certain embodiments herein can apply to analysis and structure/function/stability library construction of any protein or small molecule or other target agent with a corresponding screen or selection for activity or selection for or identification of other distinguishable characteristic. In some embodiments, with respect to a target polypeptide, library size depends on the number (N) of amino acids in a protein of interest, with a full saturation library (e.g. all 20 amino acids or non-naturally-occurring amino acids at each position) scaling as for example, 19 (or more)×N and an alanine-mapping library scaling as 1×N. Therefore, screening of even very large proteins of more than 1,000 amino acids is tractable given current multiplex oligo synthesis capabilities (e.g. 120,000 oligos). In addition to activity screens, more general properties with developed high-throughput screens and selections could be efficiently tested using these libraries. For example, universal protein folding and solubility reporters have been engineered for expression in the cytoplasm, periplasm, and the inner membrane. Moreover, due to the designed single nature of mutations (e.g., no background mutations) screening of the same protein library under different conditions (e.g., different temperatures, different substrates or co-factors, etc.) permits identification of residue changes required for expression of various traits (design criteria). In other embodiments, because residues are analyzed one at a time, mutations at residues important for a particular trait (e.g., thermostability, resistant to environmental pressures, increased or decrease in functionality or production) can be combined via multiplex recombineering with mutations important for various other traits (e.g., catalytic activity) to create combinatorial libraries for multi-trait optimization.

In certain embodiments, methods for creating and/or evaluating comprehensive, in vivo, mutational libraries of one or more target protein(s) has been described. These embodiments can be extended via a barcode tracking technology to generate trackable mutational libraries for every residue or every module in a protein. Further, embodiments disclosed herein can be based on protein sequence-activity relationship mapping method extended to work in vivo, capable of working on a few to hundreds of proteins simultaneously depending on the technology selected. For example, these methods allow mapping in a single experiment all possible residue or module changes over a collection of desired proteins for a trait of interest, as part of individual proteins of interest or as part of a pathway. Constructs and methods disclosed herein can be used for, but are not limited to, mapping i) all residue changes for all proteins in a specific biochemical pathway (e.g., lycopene production) or that catalyze similar reactions (e.g., dehydrogenases or other enzymes of a pathway of use to produce a desired effect or produce a product) or ii) all residues in the regulatory sites of all proteins with a specific regulon (e.g., heat shock response) or iii) all residues of a biological agent used to treat a health condition (e.g. insulin, a growth factor (HCG), an anti-cancer biologic, a replacement protein for a deficient population, a replacement agent for a genetic modification or dysfunction, etc.).

Certain embodiments concern assigning scores related to various input parameters in order to generate one or more composite score(s) for designing genomically-engineered organisms or systems. These scores can reflect quality of genetic variations in genes or genetic loci as they relate to selection of an organism or design of an organism for a predetermined production, trait or traits. Certain organisms or systems can be designed based on need for improved organisms for biorefining, biomass (e.g., crops, trees, grasses, crop residues, and forest residues), biofuel production and using biological conversion, fermentation, chemical conversion and catalysis to generate and use compounds, biopharmaceutical production and biologic production. In certain embodiments, this can be accomplished by modulating growth or production of microorganism through genetic manipulations disclosed herein.

Certain embodiments concern the generation and use of one or more linker amino acid sequence(s). These linker sequences need to be capable of linking a selected catalytic unit to another selected catalytic unit and/or capable of linking one module (composed of two or more catalytic units) to another catalytic unit or module. These linker amino acid sequences can reflect particular characteristics necessary in the function of the linker protein, including the ability to permit the two connected catalytic units or modules to properly maintain their tertiary structure or protein folding to conserve their naturally-occurring function or purpose. As such, these linker sequences need to be long enough to maintain separation between the catalytic units or modules but not too long to be bulky and/or interfere with the proper folding or functioning of the catalytic units or modules. These linker sequences also need to be capable of putting the catalytic units or modules to perform their desired catalytic function(s). These desired catalytic functions may be normal to the catalytic unit or module or may be or mutated. In certain embodiments, the linkers can be context-independent, wherein the same linker amino acid sequence can be used as part of multiple different modules. In certain embodiments, these linkers contain conserved regions and variable regions. In some embodiments, the linker sequences have conservation with respect to their lengths. In some embodiments, subsections of the linker sequences have conserved regions, particularly within linker classes (e.g. KS-AT linkers, AT-DH linkers. In some embodiments, the linker sequences can contain non-naturally occurring amino acids. In some embodiments the linker sequences code for linker proteins having structural conservation, in some embodiments this is conserved secondary structure, and in some embodiments this is conserved tertiary structure, and in some embodiments this is conserved secondary and tertiary structure.

In certain embodiments, domain linker sequences can be about 10 to about 500 amino acids in length. In other embodiments, domain linker sequences can be about 10 to about 450 amino acids in length. In yet other embodiments, domain linker sequences can be categorized into a linker type. In certain embodiments, the linkers categorized as Ketosynthase-Acyltransferase (KS-AT) have a length of about 5 to about 250 amino acids; in some embodiments, linker length can be about 5 to about 50 amino acids (e.g. 9 amino acids); in in some embodiments linker length can be about 5 to about 15 amino acids (e.g. 12 amino acids); in in some embodiments, the linker length can be about 10 to about 40 amino acids (e.g. 31 amino acids); in in some embodiments the length can be about 35 to 50 amino acids (e.g. 43 or 46 amino acids); in some embodiments, linker length can be about 50 to about 150 amino acids in length (e.g. 96 amino acids; 100 amino acids).

In certain embodiments, linkers can be named and categorized by the catalytic domains of which they connect together in a construct, a modular megasynthase. In accordance with these embodiments, linkers can be categorized as Acyltransferase Domain-Dehydratase Domain (AT-DH) having a length of about 50 amino acids to about 110 amino acids. In yet other embodiments, a domain linker sequence can be about 40 to about 80 amino acids in length. For example, a domain linker can be about 60 to 70 amino acids in length. In certain embodiments, domain linkers categorized as Dehydratase Domain-Enoylreductase Domain (DH-ER) can be about 150 amino acids to about 750 amino acids in length. In other embodiments, domain linkers categorized as Dehydratase Domain-Enoylreductase Domain (DH-ER) can be about 250 to about 400 amino acids in length. In certain embodiments, domain linkers categorized as Dehydratase Domain-Enoylreductase Domain (DH-ER) can be about 5 to about 75 amino acids in length; or about 10; or about 20; or about 30 amino acids in length. In certain embodiments, domain linkers categorized as Ketoreducatse Domain-Acyl Carrier Protein (KR-ACP) can be about 25 to about 400 amino acids in length. In other embodiments, domain linkers categorized as Ketoreducatse Domain-Acyl Carrier Protein (KR-ACP) can be about 50 to about 150 amino acids in length.

In certain embodiments, a construct can be a modular megasynthase containing both linkers and catalytic domains. In some embodiments, a polynucleotide encoding a modular megasynthase can have a length of about 8,000 bp to about 15,000 bp. In some embodiments, a modular megasynthase can have a length of about 2,000 amino acids to about 5,000 amino acids. In some embodiments, a modular megasynthase can be Ac-Mal_{H}-Mal_{OH} having a length of about 3500 to 4500 amino acids (e.g. 4,312 amino acids, encoded by a polynucleotide having a length of about 12,939 bp). In some embodiments, a modular megasynthase can be Ac-Mal_{OH}-Malo_{H} having a length of about 3500 to 4500 amino acids (e.g. 3,761 amino acids, encoded by a polynucleotide having a length of about 11,286 bp). In some embodiments, a modular megasynthase can be Ac-Mal_{H}-Mal_{H} having a length of about 4000 to 5500 amino acids (e.g. 4,863 amino acids, encoded by a polynucleotide having a length of about 14,592 base pairs (bp.)). In some embodiments, a modular megasynthase can be Ac-Mal_{OH}-Mal_{H} having a length of about 4,312 amino acids, encoded by a polynucleotide having a length of about 10,029 bp.). In some embodiments, a modular megasynthase can be Ac-Ser-Malo_{H} having a length of about 3500 to 4500 amino acids (e.g. 3,342 amino acids, encoded by a polynucleotide having a length of about 10,029 bp.). In some embodiments, a modular megasynthase can be Ac-Ser-Mal_{H} having a length of about 3500 to 4500 amino acids (e.g. 3,893 amino acids, encoded by a polynucleotide having a length of about 11,682 bp.). In some embodiments, a modular megasynthase can be Ile-Mal_{OH}-Malo_{H} having a length of about 3500 to 4500 amino acids (e.g. 3,865 amino acids, encoded by a polynucleotide having a length of about 11,598 bp.). In some embodiments, a modular megasynthase can be Ile-Mal_{OH}-Mal_{H} having a length of about 4000 to 5000 amino acids (e.g. 4,416 amino acids, encoded by a polynucleotide having a length of about 13,251 bp.). In some embodiments, a modular megasynthase can be Ile-Mal_{H}-Mal_{H} having a length of about 4000 to 5500 amino acids (e.g. 4,968 amino acids, encoded by a polynucleotide having a length of about 14,904 bp.). In some embodiments, a modular megasynthase can be Ile-Mal_{H}-Malo_{H} having a length of about 4000 to 5000 amino acids (e.g. 4,416 amino acids, encoded by a polynucleotide having a length of about 13,251 bp.). In some embodiments, a modular megasynthase can be Ac-Mal_{OH}-Ser having a length of about 3000 to 4000 amino acids (e.g. 3,347 amino acids, encoded by a polynucleotide having a length of about 10,044 bp.). In some embodiments, a modular megasynthase can be Ac-Mal_{H}-Ser having a length of about 3500 to 4500 amino acids (e.g. 3,898 amino acids, encoded by a polynucleotide having a length of about 11,697 bp.). In some embodiments, a modular megasynthase can be Ile-Ser-Malo_{H} having a length of about 3000 to 4000 amino acids (e.g. 3,446 amino acids, encoded by a polynucleotide having a length of about 10,341 bp.). In some embodiments, a modular megasynthase can be Ile-Ser-Mal_{H} having a length of about 3500 to 4500 amino acids (e.g. 3,997 amino acids, encoded by a polynucleotide having a length of about 11,994 bp.). In some embodiments, a modular megasynthase can be Ac-Ser-Ser having a length of about 2500 to 3500 amino acids (e.g. 2,928 amino acids, encoded by a polynucleotide having a length of about 8,787 bp.). In some embodiments, a modular megasynthase can be Ile-Mal_{OH}-Ser having a length of about 3000 to 4000 amino acids (e.g. 3,451 amino acids, encoded by a polynucleotide having a length of about 10,356 bp.). In some embodiments, a modular megasynthase can be Ile-Mal_{H}-Ser having a length of about 3500 to 4500 amino acids (e.g. 4,002 amino acids, encoded by a polynucleotide having a length of about 12,009 bp.). In some embodiments, a modular megasynthase can be Ile-Ser-Ser having a length of about 2500 to 3500 amino acids (e.g. 3,032 amino acids, encoded by a polynucleotide having a length of about 9,099 bp.).

Nucleic Acids

A “nucleic acid” can include single-stranded and/or double-stranded molecules, as well as DNA, RNA, chemically modified nucleic acids and nucleic acid analogs. It is contemplated that a nucleic acid can be of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000 or greater nucleotide residues in length, up to a full length protein encoding or regulatory genetic element.

### Construction of Nucleic Acids

Isolated nucleic acids can be made by any method known in the art, for example using standard recombinant methods, synthetic techniques, or combinations thereof. In some embodiments, the nucleic acids can be cloned, amplified, or otherwise constructed.

For example, a multi-cloning site comprising one or more endonuclease restriction sites can be added. A nucleic acid can be attached to a vector, adapter, or linker for cloning of a nucleic acid. Additional sequences can be added to such cloning and sequences to optimize their function, to aid in isolation of the nucleic acid, or to improve the introduction of the nucleic acid into a cell. Use of cloning vectors, expression vectors, adapters, and linkers is well known in the art.

### Recombinant Methods for Constructing Nucleic Acids

Isolated nucleic acids can be obtained from bacterial or other sources using any number of cloning methodologies known in the art. In some embodiments, oligonucleotide probes which selectively hybridize, under stringent conditions, to the nucleic acids of a bacterial organism. Methods for construction of nucleic acid libraries are known and any such known methods can be used.

### Nucleic Acid Amplification

Nucleic acids of interest can also be amplified using a variety of known amplification techniques. For instance, polymerase chain reaction (PCR) technology can be used to amplify target sequences directly from bacterial RNA or cDNA. PCR and other in vitro amplification methods can also be useful, for example, to clone nucleic acid sequences, to make nucleic acids to use as probes for detecting the presence of a target nucleic acid in samples, for nucleic acid sequencing, or for other purposes.

### Synthetic Methods for Constructing Nucleic Acids

Isolated nucleic acids can be prepared by direct chemical synthesis by methods such as the phosphotriester method, or using an automated synthesizer. Chemical synthesis generally produces a single stranded oligonucleotide. This can be converted into double stranded DNA by hybridization with a complementary sequence or by polymerization with a DNA polymerase using the single strand as a template. While chemical synthesis of DNA is best employed for sequences of about 100 bases or less, longer sequences can be obtained by the ligation of shorter sequences.

### Protein Methodologies

Any method known in the art for identifying, isolating, purifying, using and assaying activities of any target proteins are contemplated. Target proteins contemplated herein can include protein agents used to treat a human condition or to regulate processes (e.g., part of a pathway such as an enzyme) involved in disease of a human or non-human mammal. Any method known for selection and production of antibodies or antibody fragments is also contemplated.

### Computer Programs

Embodiments disclosed herein for generating a multienzyme complex of use for producing target agents can be provided as a computer program product which can include a machine-readable medium having stored thereon instructions which can be used to program a computer (or other electronic devices) to perform a process. The machine-readable medium can include, but is not limited to, floppy diskettes, optical disks, compact disc read-only memories (CD-ROMs), thumb drives, cloud storage and magneto-optical disks, ROMs, random access memories (RAMs), erasable programmable read-only memories (EPROMs), electrically erasable programmable read-only memories (EEPROMs), magnetic or optical cards, flash memory, or other type of media/machine-readable medium suitable for storing electronic instructions. Moreover, embodiments of the present disclosure can also be downloaded as a computer program product, wherein the program can be transferred from a remote computer to a requesting computer by way of data signals embodied in a carrier wave or other propagation medium via a communication link (e.g., a modem or network connection).

For the sake of illustration, various embodiments of the present invention have herein been described in the context of computer programs, physical components, and logical interactions within modem computer networks. While these embodiments describe various aspects in relation to modem computer networks and programs, methods and apparatus described herein are equally applicable to other systems, devices, and networks as one skilled in the art will appreciate. As such, the illustrated applications of the embodiments are not meant to be limiting, but instead exemplary. In addition, embodiments are applicable to all levels of computing from the personal computer to large network mainframes and servers.

The term “component” can refer broadly to a software, hardware, or firmware (or any combination thereof) component. Components are typically functional components that can generate useful data or other output using specified input(s). A component can or cannot be self-contained. An application program (also called an “application”) can include one or more components, or a component can include one or more application programs.

Some embodiments include some, all, or none of the components along with other modules or application components. Still yet, various embodiments can incorporate two or more of these components into a single module and/or associate a portion of the functionality of one or more of these components with a different component.

The term “memory” can be any device or mechanism used for storing information. In accordance with some embodiments of the present invention, memory is intended to encompass any type of, but is not limited to, volatile memory, nonvolatile memory and dynamic memory. For example, memory can be random access memory, memory storage devices, optical memory devices, magnetic media, floppy disks, magnetic tapes, hard drives, SIMMs, SDRAM, DIMMs, RDRAM, DDR RAM, SODIMMS, erasable programmable read-only memories (EPROMs), electrically erasable programmable read-only memories (EEPROMs), compact disks, DVDs, and/or the like. In accordance with some embodiments, memory can include one or more disk drives, flash drives, databases, local cache memories, processor cache memories, relational databases, flat databases, and/or the like. In addition, those of ordinary skill in the art will appreciate many additional devices and techniques for storing information can be used as memory.

Memory can be used to store instructions for running one or more applications or modules on processor. For example, memory could be used in some embodiments to house all or some of the instructions needed to execute the functionality of one or more of the modules and/or applications.

Exemplary Computer System Overview

Embodiments herein can include various steps. A variety of these steps can be performed by hardware components or can be embodied in machine-executable instructions, which can be used to cause a general-purpose or special-purpose processor programmed with the instructions to perform the steps. Alternatively, the steps can be performed by a combination of hardware, software, and/or firmware.

It is contemplated herein that components that make up a system for designing a modular multienzyme construct for production of target molecules (e.g. polypeptide, small molecules etc.) of embodiments disclosed herein can be provided as a kit. Further, organisms housing a modular multienzyme construct for production of target molecules (e.g. polypeptide, small molecules etc.) for expression or production of the target molecules can also be provided in kit form, for example, to fulfill a request or order.

The components described above are meant to exemplify some types of possibilities. In no way should the aforementioned examples limit the scope of the disclosure, as they are only exemplary embodiments.

Certain embodiments concern kits for producing modular megasynthases disclosed herein or kits for expressing or producing secondary metabolites of modular megasynthases.

Other embodiments concern kits having a host organism, one or more containers and one or more constructs disclosed herein for use in producing one or more secondary metabolite or one or more target molecule or agent.

### EXAMPLES

The following examples are included to illustrate various embodiments. It should be appreciated by those of skill in the art that the techniques disclosed in the examples that follow represent techniques discovered to function well in the practice of the claimed methods, compositions and apparatus. However, those of skill in the art should, in light of the present disclosure, appreciate that many changes can be made in the embodiments which are disclosed and still obtain a like or similar result without departing from the spirit and scope of the disclosure.

### Example 1

It is understood that megasynthases are valuable complexes for producing secondary metabolites. Previous studies demonstrate that wild-type genes of modular megasynthases were difficult to mutate or manipulate. In addition, wild-type genes were difficult to express due in part to their size and complexity. Further, due to this difficulty in expressing a wild-type megasynthase gene in a host, modulating or editing the expressed wild-type gene in the host created additional problems. For example, a wild-type gene of a megasynthase is extremely difficult to modulate or edit using in vivo editing tools. Therefore, other methods must be used to facilitate mutating and expressing these genes.

In certain methods, non-naturally occurring genes were created by (1) building functional scaffolds that can be cloned into the E. coli genome instead of high copy plasmids; (2) using codon harmonization instead of optimization to match the codon usage of E. coli; (3) using catalytic and tailoring domains from modular megasynthases that have already been proven to function in E. coli; and/or (4) linking the catalytic domains and modules. Generally, the sequences linking catalytic domains of megasynthases were considered junk DNAs as they were thought to be irrelevant to the overall function of the enzyme. However, this disclosure demonstrates that the linker sequences are essential for maintaining structure, and thus, function of catalytic domains of megasynthases.

To determine a linker sequence, a machine learning-based tool was used to build up a dataset to for developing certain designs. Medema et al., NUCL ACIDS RES (2011), released the ‘antibiotics and Secondary Metabolite Analysis SHell’ (antiSMASH). antiSMASH uses profile Hidden Markov Models (pHMMs) to discover secondary metabolite gene clusters in nucleotide sequence data. Here, antiSMASH 2.0 was used to collect every putative modular megasynthase gene (including PKS, NRPS, and PKS-NRPS hybrids) from the over 2,000 complete, annotated bacterial genomes on NCBI. Given the specific designs sought a database for appropriate linkers was probed.

In other methods, to uncover efficient methods for linking two target catalytic domains, amino acid sequences of every linker from the database of extension modules were deciphered, and categorized them per their catalytic domains linked. For example, in some exemplary methods, linkers were categorized as Ketosynthase-Acyltransferase (KS-AT). In other exemplary methods, linkers were classified as Acyl Carrier Protein-Ketosynthase (ACP-KS). Additional exemplary linker types identified using methods of this disclosure are detailed in Table 1.

TABLE 1
Exemplary Linker Types

| Linker | Catalytic Domains | | Size | Polypeptide |
|---|---|---|---|---|
| Category | Connected | Exemplary Sequence | (aa) | sequence |
| ACP | Acyl Carrier Protein- | AAIRSNVDPTGPTPLTPIQLWFFQ | 437 | SEQ ID NO: 70 |
| Condensation | Condensation Domain | QELPEPSLFTQSVLLEVPANTDA | | |
| | | ERLSTALLQLCECHAALRLRFHR | | |
| | | AKDGWQQFIPAATVPPDFETHTL | | |
| | | ATPSEMEQLTQAAEARIDIVHGP | | |
| | | LLAARLFTFSDGSPSRLFFTIHHL | | |
| | | AVDGVSWRILLEDLYRAYHQQP | | |
| | | LAPPATSFREWSLHLRDVAKSPS | | |
| | | LADEVSFWQQVPSCNLWPTQEK | | |
| | | NLVSEEASCSFELDEHATAALLR | | |
| | | QAPRTYNASIQELLVAALAQGV | | |
| | | ASTTGHSRVTLDVERHGRHASD | | |
| | | PQTDLSRTVGWFTTIYPVSVSVA | | |
| | | VSASIRDSVPSVREQLRRIPEEGF | | |
| | | HYPILRYLAAPNAFRDSQPSPILF | | |
| | | NYHGQIDTALQQTVEWKPASEI | | |
| | | VTPLRSLRARRSHLFEIISAVSNN | | |
| | | RLQVEWHYNSRLQERSAIEALAS | | |
| | | NFQQQLIALCHPPIHLQSSPAIV | | |
| ACP | Acyl Carrier Protein- | EEALNIDGLAVVHDPED | 17 | SEQ ID NO: 71 |
| Heterocyclization | Heterocyclization | | | |
| | Domain | | | |
| ACP-KS | Acyl Carrier Protein- | LGTDSKLPGGRRGTSDEP | 18 | SEQ ID NO: 73 |
| | Ketosynthase Domain | | | |
| ACP-KS | Acyl Carrier Protein- | GLPQEIKHTPAVRTTSEDP | 19 | SEQ ID NO: 74 |
| | Ketosynthase Domain | | | |
| ACP-TE | Acyl Carrier Protein- | VDTTAAETGGDTLSALFRSGLEA | 73 | SEQ ID NO: 76 |
| | Thioesterase Domain | GAVGEAYDLLRSVVRLRPRFRT | | |
| | | VDEVGDLAPAVTLRESDSELPRL | | |
| | | ICLST | | |
| ACP-TE | Acyl Carrier Protein- | TSGEDNPIPLCQGDGEE | 17 | SEQ ID NO: 77 |
| | Thioesterase Domain | | | |
| A-PCP | Adenylation Domain- | PVQVNNQTQLSAYCQTDKTLEI | 83 | SEQ ID NO: 78 |
| | Peptide Carrier Protein | AEIREFLAKFLPVYMIPSYFIFLK | | |
| | | QFPLTRHGKLDLHSLRELKETSK | | |
| | | SLVNSNYVAPRNHL | | |
| AT-ACP | Acyltransferase Domain- | KAAAPARAAKPAATPESLPFVLS | 109 | SEQ ID NO: 79 |
| | Acyl Carrier Protein | AFTVPALRAQADKLHLHMGMNI | | |
| | | QDRFLDVAYSLAFERTHFRKRL | | |
| | | VVFAKGKSDLLDALASYGRTGE | | |
| | | VPAGAVSMVDDRDRECRLAL | | |
| AT-DH | Acyltransferase Domain- | AKDETQTMQKGLAELHVLGAPV | 66 | SEQ ID NO: 81 |
| | Dehydratase Domain | DWRGFFAPYGGERVKLPTYAFQ | | |
| | | RERYWLEPMPTRAVGAGLNDAN | | |
| AT-DH | Acyltransferase Domain- | GMRREQPLPLGLRRLLTDLHNA | 69 | SEQ ID NO: 82 |
| | Dehydratase Domain | GAAVDFSVLCPQGRLVDAPLPA | | |
| | | WSHRFLFYDREGVDNRSPGGST | | |
| | | VAV | | |
| AT-KR | Acyltransferase Domain- | AHPQQRDRDAQQLAQALAGLW | 301 | SEQ ID NO: 84 |
| | Ketoreductase Domain | TAGVEIDWPATRGGARRRNVPL | | |
| | | PTYPFQRQRFWVEAGAPRPQDD | | |
| | | ASAAQPQGLYYLPAWVVQRSG | | |
| | | AVPPAAGPGPGDTVLLLGGAGL | | |
| | | PLAERLARRLAERGARVVKVAA | | |
| | | AEGFRDDGGGGFALRPEERADH | | |
| | | AALWRALGPTHAFHCWGLEED | | |
| | | WEQGYFSLLALVQGREDAGRDG | | |
| | | PLSLTIVIDRAEEVAGNEPLEPGR | | |
| | | ATVAALARVLGQEMPRIACRDV | | |
| | | LVPNAGSAAEAQLAEALAGEPA | | |
| | | RPVDEFCVAWRGAQRWVKTYE | | |
| | | PVPAPAACAQRLRQGS | | |
| AT-KR | Acyltransferase Domain- | ADHRQITEAVADAYVLGHLPDF | 342 | SEQ ID NO: 85 |
| | Ketoreductase Domain | AALRRPHARKLDLPTYPFERRQY | | |
| | | WFRDARERPEQPRDTGGPRTEA | | |
| | | VRLLEDGRIEELAALLGGASDDQ | | |
| | | HTLQVLTKLAAQHNQQRTTRSI | | |
| | | ADDRYEIRWDRSTSPLSGADVD | | |
| | | QAGSWIVVSDDADAVPPLVDLL | | |
| | | AARNEPHQVVGLPASDADEERL | | |
| | | AETLRAAATEDATLRIVHVAALE | | |
| | | AGATPSMRSLLRMQHRILGGTR | | |
| | | RVFRAAVAAELRGPIWIVTRGA | | |
| | | QRVADTDTVAPHQSCLWGFGRA | | |
| | | ASLELPHVWGGLADLSEGGDNA | | |
| | | ADEWSALVDRIAAPHGSAVRED | | |
| | | QLALRDRAVYTPRLARRSAPPSG | | |
| | | TPLHLRGD | | |
| Condensation A | Condensation Domain- | TTSFIEHLQHTKQTLIDAFRHQSY | 183 | SEQ ID NO: 87 |
| | Adenylation Domain | TLEDLLAALQLPRDFSRSPLISVS | | |
| | | FNMDPSLTLPEFKDLKVSLPPSPI | | |
| | | SYTPFDLGFNLIELNDNLIIYCNY | | |
| | | NTELFKKETIKQFLESFEILLRGII | | |
| | | DDANHLLYQLPLLTPVQQEKLL | | |
| | | RQLTGKTRKLPEKATIIDDFVAQ | | |
| | | VKLTPNAPALIAGKISL | | |
| DH-ER | Dehydratase Domain- | AAWHVDGDVWARVALPEAAAS | 336 | SEQ ID NO: 88 |
| | Enoylreductase Domain | SAIRYGLHPALLDSSMHSLLLTQ | | |
| | | RLKAQVGDDVFVPFEAERLSVW | | |
| | | KDGLAEVWVKVAEFELGEGEF | | |
| | | WASLDLYDTSGEHVGRLQRLHA | | |
| | | RRIDRAALRRLAAAGVDRFLFRT | | |
| | | EWQPVEAPEAVFGGTWGLLGG | | |
| | | ADAPWADEVRSRLMQAGAQVV | | |
| | | DIARLSEAEACDGVIQLWGGDG | | |
| | | QVVESSHRQAASALVQVQELAL | | |
| | | AGFAKPVIWLTRGAVGTSSDDP | | |
| | | VSDLGASPLWGLLRTARNEHPE | | |
| | | LALRIVDLGDTAADLDTLASALA | | |
| | | LADEPECALRGAKVLAPRLKKA | | |
| | | PANAGLVLPAEGNWRLEIATKG | | |
| | | RLDQPLS | | |
| DH-ER | Dehydratase Domain- | VAYVAEDATATMLAEVALPGSI | 357 | SEQ ID NO: 89 |
| | Enoylreductase Domain | RSQQGLYAIHPALLDACFQSVGA | | |
| | | HPDSQSVGSGLLVPLGVRRVRA | | |
| | | YAPVRTARYCYTRVTKVELAGV | | |
| | | EADIDVLDAHGTVLLAVCGLRIG | | |
| | | TGVSERDKHNRVLNERLLTIEW | | |
| | | HQRELPEMDPSGAGKWLLISDC | | |
| | | AASDVTATRLADAFREHSAACT | | |
| | | TMRWPLHDDQLAAADQLRDQV | | |
| | | GSDEFSGVVVLTGPNTGTPHQGS | | |
| | | ADRGAEYVRRLVGIARELSDLPG | | |
| | | AVPRMYVVTRGAQRVLADDCV | | |
| | | NLEQGGLRGLLRTIGAEHPHLRA | | |
| | | TQIDVDEQTGVEQLARQLLATSE | | |
| | | EDETAWRDNEWYVARLCPTPLR | | |
| | | PQERRTIVADHQQSGMRLQIRTP | | |
| DH-KR | Dehydratase Domain- | AAWRQGDVVYADVRLPVPDGA | 241 | SEQ ID NO: 90 |
| | Ketoreductase Domain | EGLHPALLDAALHPARLLDDRD | | |
| | | RTPRMPFLWAGVHRYDGGATQ | | |
| | | ARVRIMRAGGHSAEQIAVQLAG | | |
| | | PDGRALFEVEALTVRPVPRSVHQ | | |
| | | PTWVTVTPPATVPVEPGVLDLSG | | |
| | | PPAHSPEEVRDQVWEAAEKLRS | | |
| | | RLPGPRIVVVTRSAAVAGLIRVAI | | |
| | | TEYPGQVALVEWDGGAASDHV | | |
| | | LPAAIRAAATAPEIRIADGRIGSP | | |
| | | RLVRAAIAGPGTGGFGD | | |
| Dual | Dual | AAWHVDGDVWARVALPEAAAS | 186 | SEQ ID NO: 91 |
| Condensation A | Condensation/Epimeri- | SAIRYGLHPALLDSSMHSLLLTQ | | |
| | zation Domain- | RLKAQVGDDVFVPFEAERLSVK | | |
| | Adenylation Domain | DGLAEVWVKVAEFELGEGEFW | | |
| | | ASLDLYDTSGEHVGRLQRLHAR | | |
| | | RIDRAALRRLAAAGVDRFLFRTE | | |
| | | WQPVEAPEAVFGGTWGLLGGA | | |
| | | DAPWADEVRSRLMQAGAQVVD | | |
| | | IARLSEAEACDGVIQLWGGDGQ | | |
| | | VVESSHRQAASALVQVQELALA | | |
| | | GFAKPVIWLTRGAVGTSSDDPVS | | |
| | | DLGASPLWGLLRTARNEHPELA | | |
| | | LRIVDLGDTAADLDTLASALALA | | |
| | | DEPECALRGAKVLAPRLKKAPA | | |
| | | NAGLVLPAEGNWRLEIATKGRL | | |
| | | DQPLS | | |
| ER-KR | Enoylreductase Domain- | PTQRRGMVDPD | 11 | SEQ ID NO: 93 |
| | Ketoreductase Domain | | | |
| ER-KR | Enoylreductase Domain- | IPQTGKSLVTLPPEQAQVFRPD | 22 | SEQ ID NO: 94 |
| | Ketoreductase Domain | | | |
| Heterocyclization | Heterocyclization | PVAAFGERLATLHGRLWQDLDH | 183 | SEQ ID NO: 95 |
| A | Domain-Adenylation | RLCGGVEVLREIARRRGRAAAA | | |
| | Domain | LPVTFTSTVSGAPTPGAGLMPGA | | |
| | | RLRYGISQTPQVWIDCQMMAED | | |
| | | GGLLLHWDVRDGVLPDGVAAD | | |
| | | MFAAFTELVERLADGDAVDEAD | | |
| | | PVVLPRRQRELVAAANDTAEPR | | |
| | | VRGPLHAAFLDRARRDPGRVAV | | |
| | | IAAGCTL | | |
| Joint AC-PC | (Both Acyl and Peptide) | LGTDSKLPGGRRGTSDEP | 18 | SEQ ID NO: 96 |
| | Carrier Protein- | | | |
| | Condensation Domain | | | |
| KR-ACP | Ketoreducatse Domain- | QGMAARLSDADKTRFSRQGME | 109 | SEQ ID NO: 98 |
| | Acyl Carrier Protein | ALGPTEALDLFEAAVMSDAPMA | | |
| | | VAAALDLGRLQRTLEDNNGGSA | | |
| | | PALYRELFSRAAGGR | | |
| | | GAGGGAGGGAGLRKLLVETAV | | |
| | | EQREAAVL | | |
| KR-ACP | Ketoreducatse Domain- | IGRAIAFAEQTGDAIAPEEGAYA | 92 | SEQ ID NO: 99 |
| | Acyl Carrier Protein | FETLLRHNRAYSGYAPVIGSPWL | | |
| | | TAFAQHSPFAEKFQSLGQNRSGT | | |
| | | SKFLAELVDLPREEWPDRLRRLL | | |
| KS-AT | Ketosynthase Domain- | QEVRPAPGQGLSPAVSTLVVAG | 106 | SEQ ID NO: 101 |
| | Acyltransferase Domain | KTMQRVSATAGMLADWMEGPG | | |
| | | ADVALADVAHTLNHHRSRQPKF | | |
| | | GTVVARDRTQAIAGLRALAAGQ | | |
| | | HAPGVVNPAEGSPGPGTVF | | |
| KS-AT | Ketosynthase Domain- | KAAAPARAAKPAATPESLPFVLS | 109 | SEQ ID NO: 102 |
| | Acyltransferase Domain | AFTVPALRAQADKLHLHMGMNI | | |
| | | QDRFLDVAYSLATTRTHFRKRL | | |
| | | VVFAKGKSDLLDALASYGRTGE | | |
| | | VPAGAVSMVDDRDECRLAL | | |
| Conserved Motif | Conserved Motif | AEAGPEPERGPVPAVSTLVVFGK | 100 | SEQ ID NO: 103 |
| KS-AT | Ketosynthase Domain- | TAQRVAATASVLADWMEGPGA | | |
| | Acyltransferase Domain | EVALADVAHTLNHHRARQTRFG | | |
| | | TVVARDRAQAIAGLRALAAGQH | | |
| | | APGVVAPREGSP | | |
| Conserved Motif | Conserved Motif | LADVAHTLNHHRARHAKFATVC | 28 | SEQ ID NO: 104 |
| KS-AT | Ketosynthase Domain- | ARDRAQ | | |
| | Acyltransferase Domain | | | |
| Conserved Motif | Conserved Motif | PAVSTLVVSGKTPERIASTAGAL | 31 | SEQ ID NO: 105 |
| KS-AT | Ketosynthase Domain- | ADWLAGPG | | |
| | Acyltransferase Domain | | | |
| Conserved Motif | Conserved Motif | PGTVF | 5 | SEQ ID NO: 106 |
| KS-AT | Ketosynthase Domain- | | | |
| | Acyltransferase Domain | | | |
| Conserved Motif | Conserved Motif | AVAGLRALAAGQPAPGVVGPHD | 22 | SEQ ID NO: 107 |
| KS-AT | Ketosynthase Domain- | | | |
| | Acyltransferase Domain | | | |

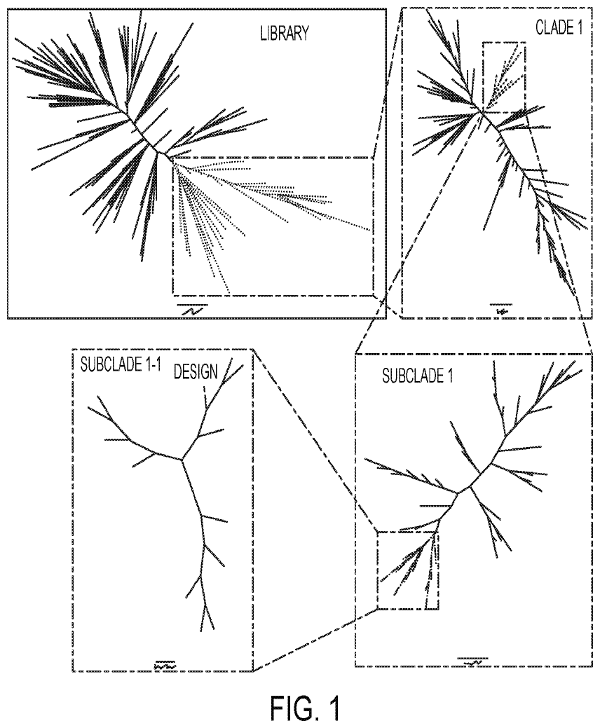

Biological diversity analyses the linker databases found conserved patterns. Using KS-AT linkers as an example, the KS-AT linker database analyzed contained 2,614 individual genes. A histogram of the lengths of the KS-AT linkers yielded unimodal results with a mean length of 106, indicating a conserved pattern. In addition, a calculation of Simpson's measure of evenness (E_{D}) was 0.283, indicating that some of the linkers in the KS-AT database contained identical sequences. Further analysis found only 1,876 of the 2,614 linkers were unique, indicating that 738 linker sequences are identical to one or more other KS-AT linkers (across different bacterial species and enzyme contexts). To determine the frequency of these identical (e.g. overrepresented) linkers, a rank-abundance (Whittaker) plot was generated. If each species were equally represented, this plot would have yielded a flat result, with each species (1/2614) representing 0.038% of the database. In these results, the mean abundance was 0.065% with standard deviation of 0.11%. 23 species had abundances over 2 standard deviations above the mean, with the most abundant at 2.14%, or a 46× enrichment. This determined that each linker database was biased toward a small number of Operational Taxonomic Units (OTUs). (See for example, FIG. 1).

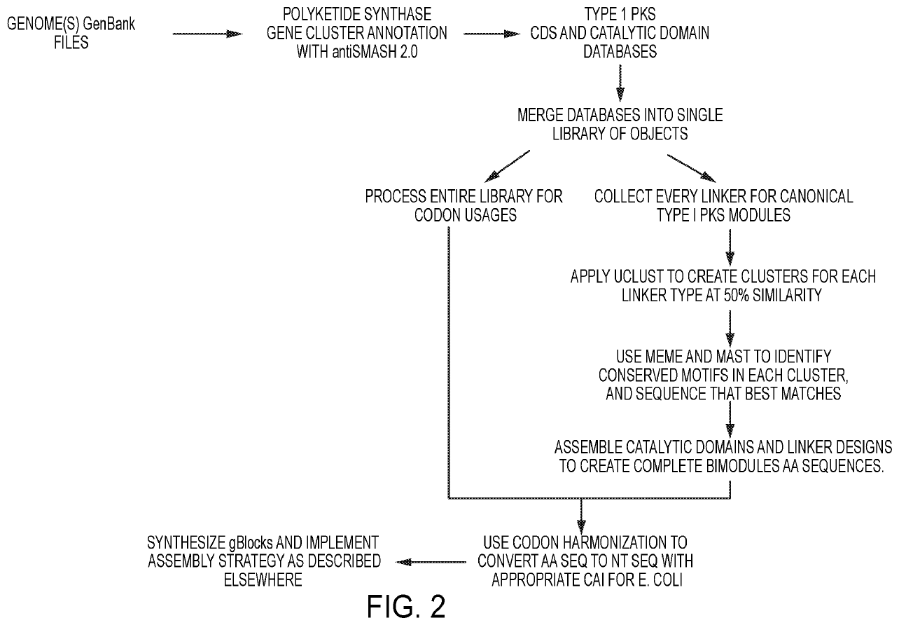

To identify one possible design for each linker class, sequences were clustered at a level of 50% similarity to identify all sequences in the given database with high similarity to the enriched OTU. Then, the largest cluster in a set was selected, and clustering tools, as known and used by those of skill in the art (Multiple Expectation Maximization for Motif Elicitation (MEME) and Motif Alignment and Search Tool (MAST)) were performed to score each sequence for representativeness of the cluster. The sequence with the highest score was selected. Functional genes including two or more catalytic domains and a linker amino acid sequence were generated by combining the domains and the linker in an appropriate order (see for example, FIG. 2).

To process the information generated in the pattern processing linker dataset and yield a linker design, clustering analysis was implemented in UCLUST. (Edgar, 2010) Clustering identified the ‘biased’ OTU all other linkers within the database with sequence homology to the OTU.

In another method and as detailed above, using KS-AT as an exemplary linker design, the complete 2,614 sequence database was divided into 961 clusters with a mean relative abundance of 0.1%. 17 clusters were over 2 standard deviations above the mean relative abundance. The largest cluster contained 117 sequences, or 5.45% of the entire database, an almost 55× enrichment. Using the largest 117-member cluster from the database a scoring methodology was used to design a single exemplary KS-AT linker. MEME analysis (a form of machine learning) expectation maximization can be used to identify the conserved sequence motifs within a set of sequences. Given a set of motifs (such as those generated by MEME) and a target sequence, MAST calculates the statistical significance of motif matches to the target sequence, and as such, was an ideal way to profile the conserved sequences within the cluster, and assign each sequence a score (i.e. statistical significance). Using this method, the cluster was analyzed for conserved motifs using MEME, and MAST was then used to score each member of the subset. MEME identified five conserved motifs. See Table 1 and SEQ ID NOs: 102-106. MAST scored each sequence, with the highest scoring sequence a single “common” sequence was selected based on its E-value of 1.110-11. The exemplary KS-AT linker amino acid sequence designed was: QEVRPAPGQGLSPAV STLVVAGKTMQRV SATAGMLADWMEGPGADVALADVAHT LNHHRSRQPKFGTVVARDRTQAIAGLRALAAGQHAPGVVNPAEGSPGPGTVF (SEQ ID NO: 101). This amino acid sequence was used as the final KS-AT linker design. The same approach was also applied to identify designs for all other linker classes. See Table 1.

In the past, proper folding of a target polypeptide has been a significant problem when expressing modular megasynthases to produce a target polypeptide in E. coli.

Further, codon optimization can be used to maximize heterologous protein expression; however, codon usage can have an important effect in RNA secondary structure, gene expression, and/or protein folding. Given the importance of codon usage in protein folding, codon harmonization is used to match sequences of non-native to that of native organisms. In addition, genes produced using the methods disclosed herein can include lac operators for IPTG-inducible expression.

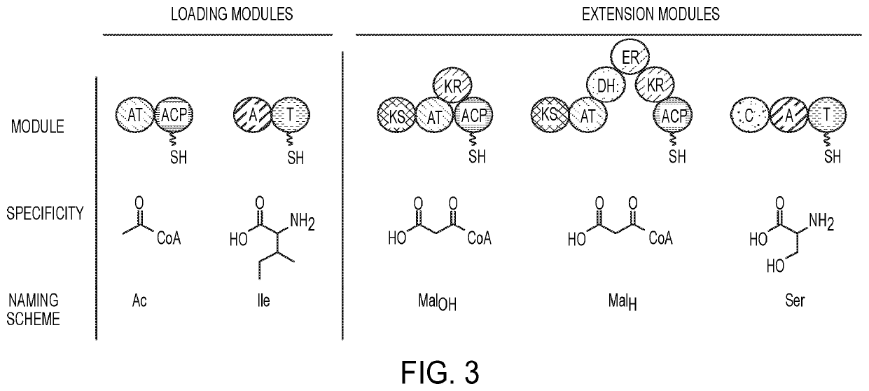

In certain exemplary methods, to validate the designed functional domains and linker amino acid sequence, SWISS-MODEL was used to homology model the structure of each sequence. (See for example, Arnold, K., Bordoli, L., Kopp, J. & Schwede, T. (The SWISS-MODEL workspace is a web-based environment for protein structure homology modelling. Bioinformatics 22, 195-201, 2006). An exemplary model of the catalytic molecules and modules for use with a KS-AT linker is illustrated in FIG. 3. The KS-AT linker model sequence aligned to a control KS-AT linker that was crystallized as part of a crystal structure of the Acyltransferase isolated from Mycobacterium tuberculosis. This type of validation was performed for each linker design of appropriate size (>30 amino acids in length) (data not shown).

### Example 2

In other exemplary methods, an exemplary non-naturally occurring (“synthetic”) gene encoding a polyketide synthase (PKS) was created to produce a target small molecule, delta-hexalactone, which is a food and flavor ingredient. The computational tool assembled amino acid sequences of the target catalytic domains and linkers (see for example, FIG. 2). A selected amino acid sequence was codon harmonized and synthesized in fragments by GenScript. These fragments were assembled into a complete gene via yeast TAR cloning. This synthetic gene was then restriction digested out of its associated plasmid, and this linear piece was integrated into the E. coli genome. Expression was induced with IPTG, and then shotgun proteomics and metabolomics were performed.

The initial genome mining of NCBI identified 6,943 possible Type I PKS coding sequences. These coding sequences (CDSs) were filtered to eliminate duplicates, and 2,837 extension modules that follow the canonical PKS logic were collected in the database. Codon Adaptation Indices (CAI) were calculated for each PKS gene discovered to determine any patterns that might exist in codon usage. It appeared that the codon usage in PKS genes do not drastically differ from codon usage across the genome, generating a Median CAI of 0.697 (data not shown).

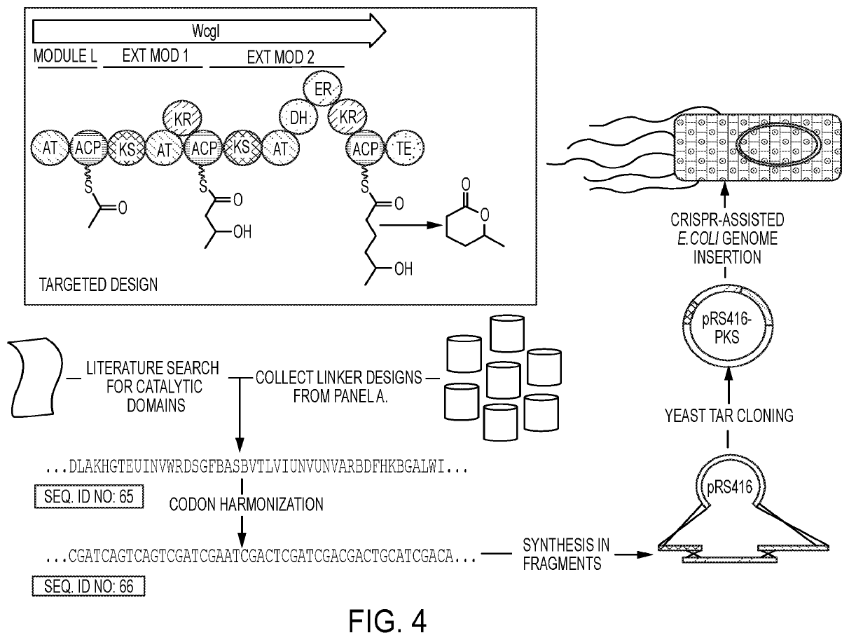

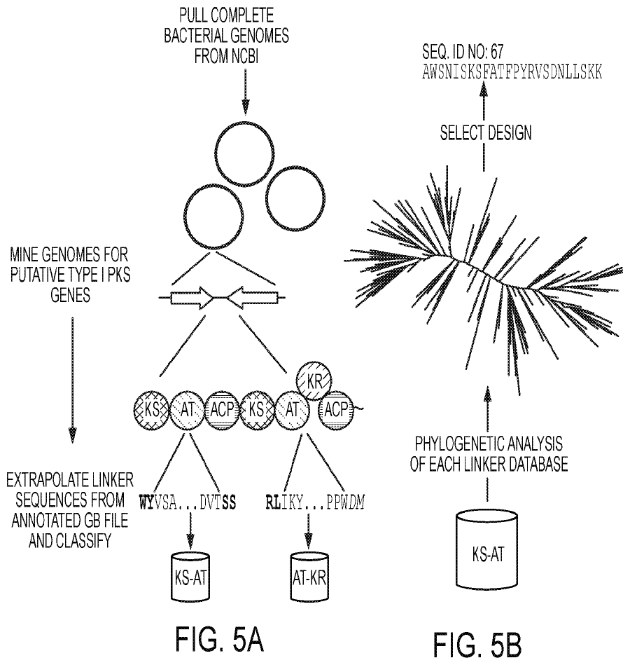

FIG. 4 represents a gene design and assembly. As a proof of concept, a target product (e.g., delta-hexalactone) from available synthetic standards was chosen. Given this target, a non-naturally occurring PKS was designed using catalytic domains shown in the literature to function in E. coli and interdomain linkers from FIG. 5A. The target gene shown in FIG. 4 has the following structure: AT-ACP-KS-AT-KR-ACP-KS-AT-DH-ER-KR-ACP-TE. Amino acid sequence was codon harmonized for E. coli, assembled via TAR-cloning in S. cerevisiae, and inserted into the genome of E. coli strain BL21(dE3). (SEQ ID NOS: 65 and 66).

FIG. 5B represents a computational mining and a linker design. Briefly, bacterial genomes for putative Type I PKS, NRPS, and hybrid gene clusters were mined. Inter-domain linker sequences from putative clusters were determined, and linkers based on flanking domains were classified. Each class of linkers was clustered, and a final linker design from the largest cluster was selected.

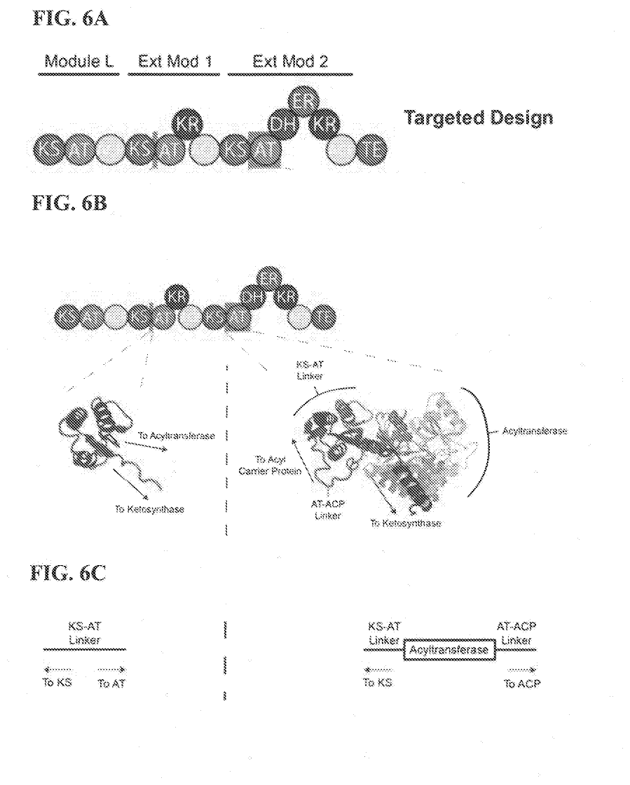

All the linker designs selected had structural similarities to linkers in known crystal structures of control molecules (see for example, FIGS. 6A-C). These linkers are highly similar in structure, with this design appearing to be slightly less rigid. The gene was completely sequenced and contained only two mutations, both of which were non-synonymous (data not shown).

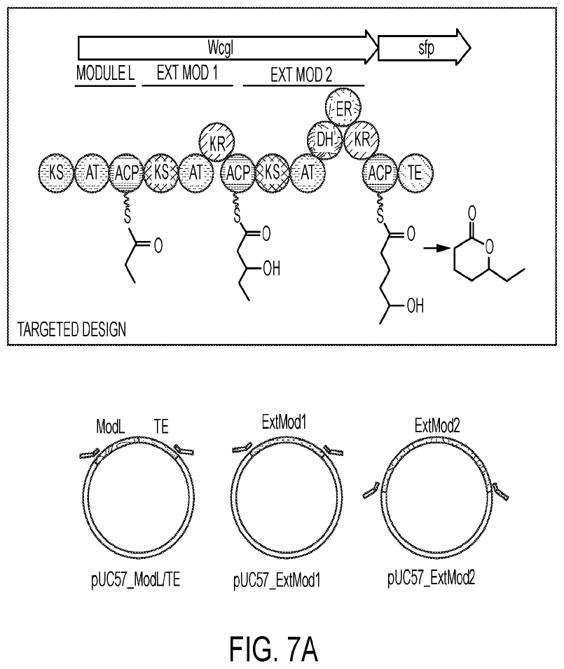

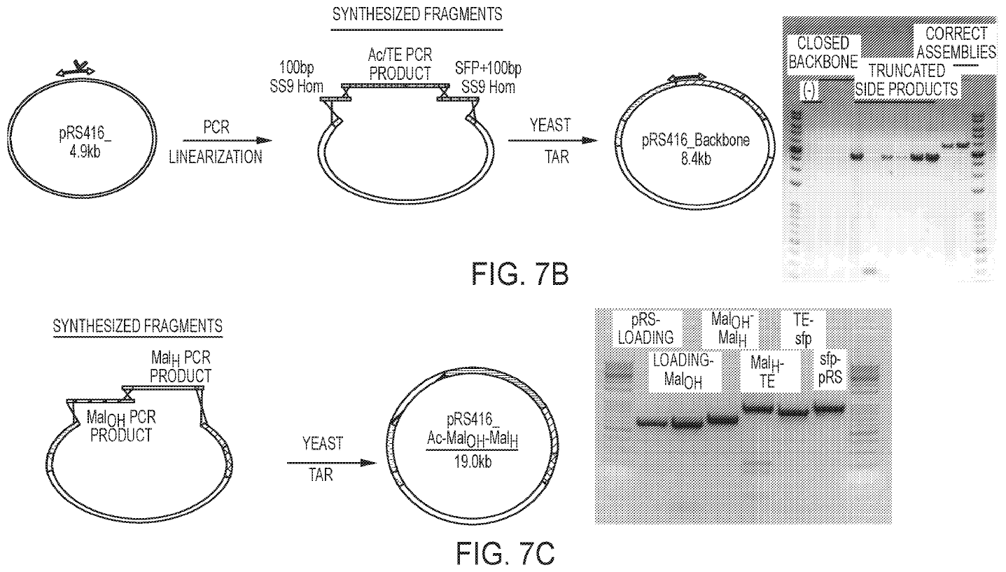

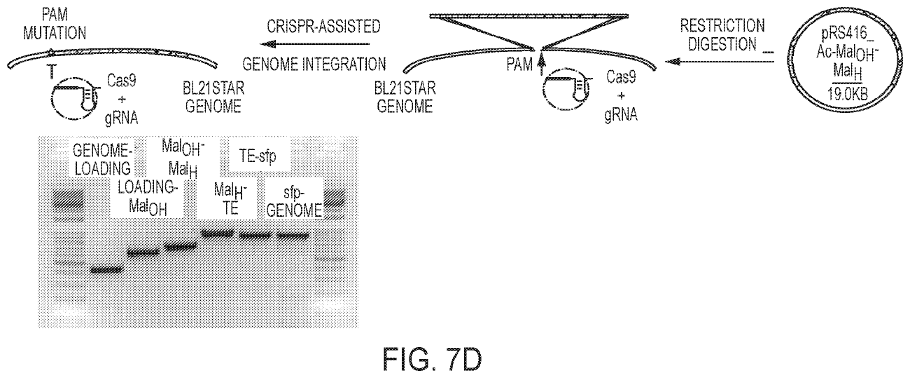

In this example, the gene was codon harmonized and synthesized in fragments by GenScript (see for example, FIG. 7A). Yeast TAR cloning of these fragments went as expected (see for example, FIGS. 7B and 7C). The gene was then integrated into the E. coli genome (see for example, FIG. 7D). Evidence suggests (see e.g. Wang Y, Pfeifer BA. 6-deoxyerythronolide B production through chromosomal localization of the deoxyerythronolide B synthase genes in E. coli. Metab. Eng. 2008; 10: 33-38) that genome integration of modular megasynthases yields better results in E. coli, so the construct was integrated into the genome of BL21(dE3)STAR.

With the targeted gene incorporated into the genome of the microorganism, the genome was qualitatively tested for expression and production of the target metabolite via mass spectrometry-based proteomics and metabolomics, (data not shown) indicating that the target non-native protein was highly expressed but was not significantly higher than native proteins. This is a valuable assessment of this system because dramatically overexpressed heterologous multimodular proteins often require significant amounts of chaperones for folding which can result in chaperone titration and the concomitant loss of folding and function.

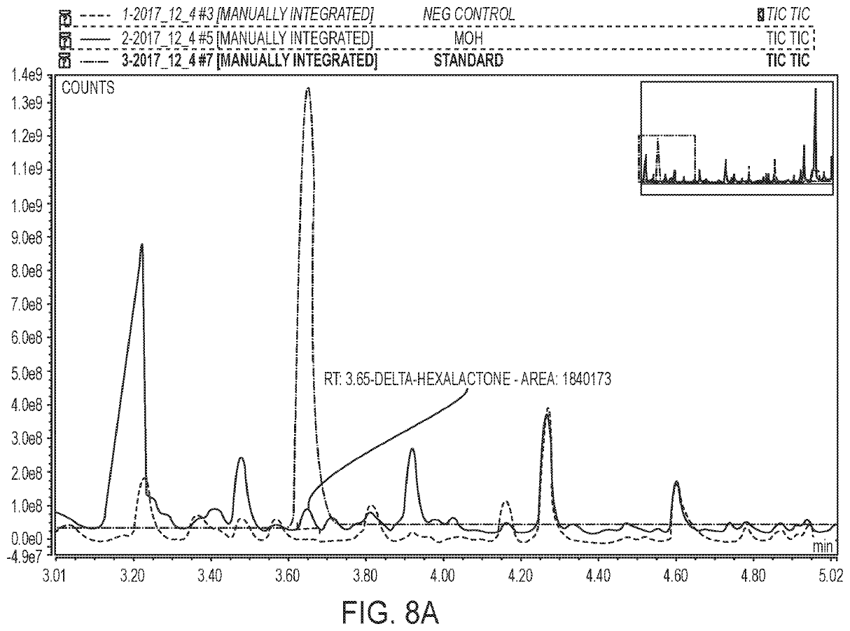

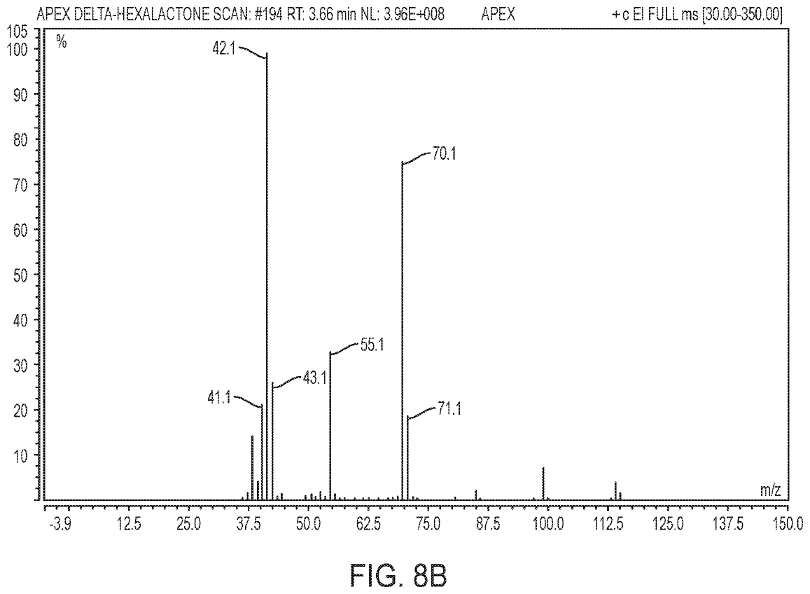

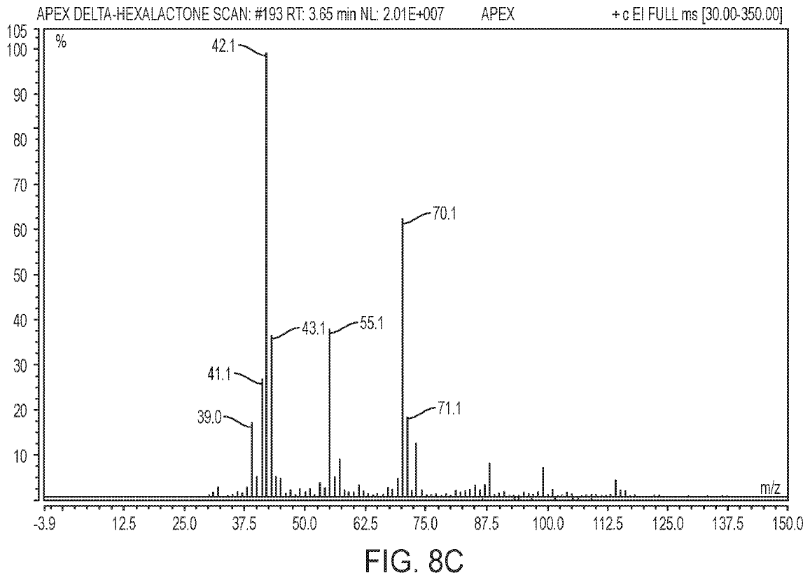

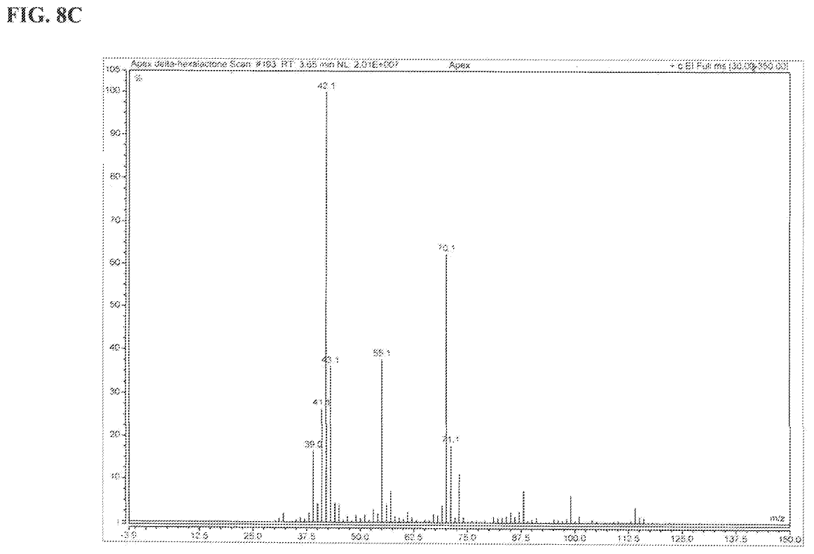

In other exemplary methods, functionality of this design was used to identify secondary metabolite of interest, Hexalactone™. For example, high cell-density fed-batch fermentations were performed and extracts of the target strain, a negative control, and the authentic standard were each analyzed via GC-MS. This demonstrated that the target gene produced the target metabolite. As illustrated in FIG. 8A, a Hexalactone™ standard had a retention time of 3.66 minutes. A negative control had no peak at 3.66 minutes, whereas there was a peak present in the extract of the engineered microorganism strain. The chromatograms from each sample are illustrated in FIGS. 8B-8C. The mass spectrum for the Hexalactone™ standard and the 3.66-minute retention time peak from this strain match. Further, a library search against the NIST MS database identified the compound at 3.66 minutes in this sample as Hexalactone™, confirming the function of this design.

### Example 3

In addition to the novel polyketide synthase (PKS) described above, the techniques described herein are applicable to other multiplex enzymes such as non-ribosomal peptide synthases (NRPSs), as well as PKS-NRPS hybrids. This example details three other novel modular megasynthases created by the instant methods: an NRPS, an NRPS-PKS hybrid with one PKS module and two NRPS modules and an NRPS-PKS hybrid two PKS modules and one NRPS module.

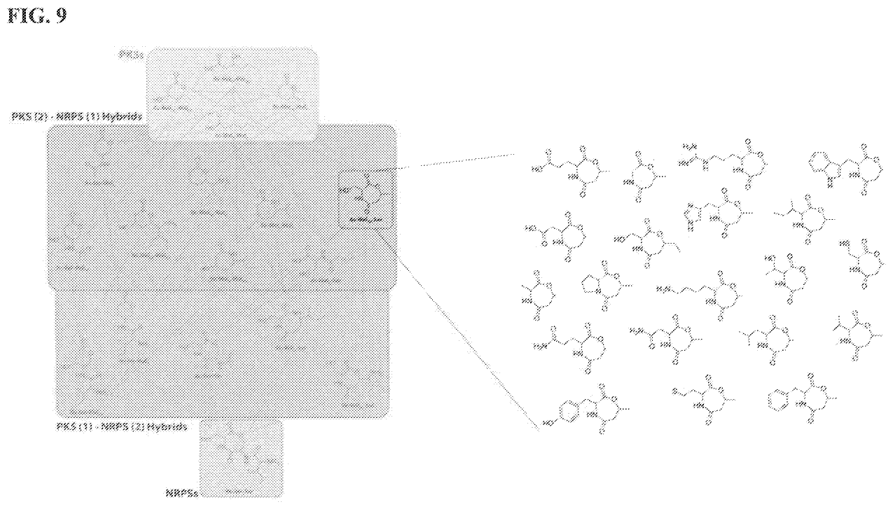

These three megasynthases are exemplified by building Ac-Mal_{OH}-Ser (using a serine-specific extension module), Ile-Mal_{OH}-Ser (using an isoleucine-specific loading module), and Ile-Ser-Ser. The structures of the molecules produced by these designs are illustrated in FIG. 9.

The design, cloning, genome integration, genome sequencing, and proteomics were performed as described in Example 2 for the 6-hexalactone-producing PKS. Results were similar to those for the 6-hexalactone-producing PKS. Each novel modular megasynthase was sequence-verified and was sufficiently expressed upon IPTG-induction, as was sfp.

As detailed in Example 2, each new strain was fermented; however, different feed mixtures were used based on required substrates for the various strain harboring the novel modular megasynthase. Unlike 8-hexalactone, the target molecules from the designs for Ac-Mal_{OH}-Ser, Ile-Mal_{OH}-Ser, and Ile-Ser-Ser presented a more difficult detection challenge due to the lack of existing authentic standards for measuring the production of the target molecules. In addition, due to increased polarity and hydrogen-bonding, it was assumed that the target molecules were unlikely to be GC compatible. As a result, an LC-MS^{N}-based metabolomics approach was performed, thereby allowing for both detection and structural validation in a single experiment.

### Example 4

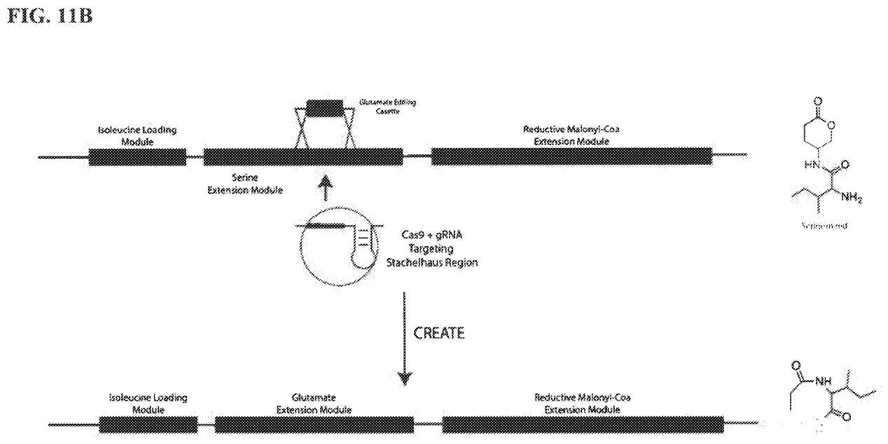

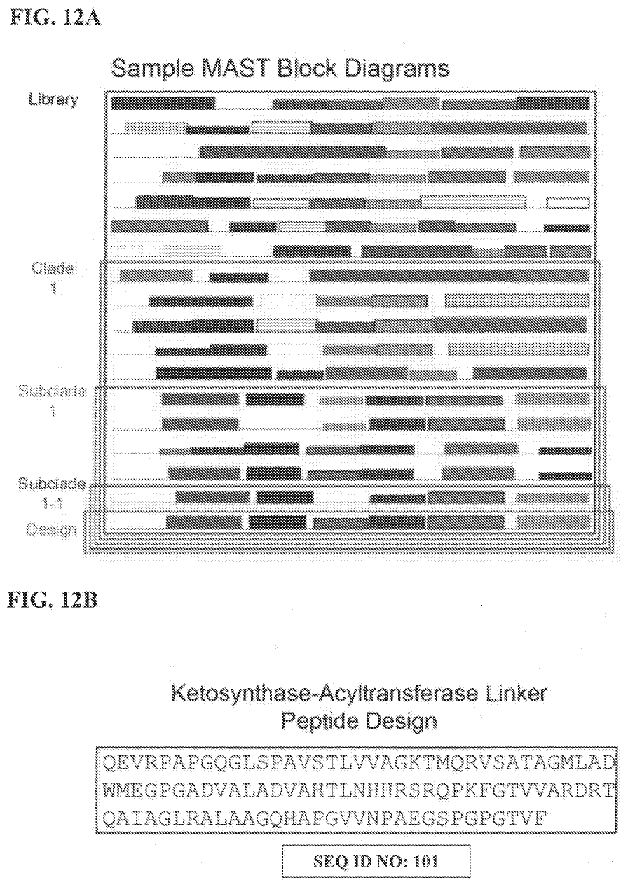

In certain exemplary methods, the principles described in the foregoing examples can be applied to the synthesis of an enormous variety of modular proteins by combining known catalytic modules with known linkers to create novel modular megasynthases. As described in Example 1, a set of desired modules has been identified, along with linker sequences, using phylogenetic analysis (FIG. 1). MEME and MAST scores (data available upon request) can be generated for each to determine a single, ideal amino acid linker sequence (FIGS. 12A and B). The selected amino acid sequence can then be codon harmonized and synthesized in fragments by for example, GenScript®. These fragments can be assembled into a complete gene via yeast TAR cloning. This synthetic gene can be restriction digested out of its associated plasmid, and this linear piece can be integrated into the E. coli genome. Expression can be induced with IPTG, and shotgun proteomics and metabolomics performed.

In this exemplary method, using PKSs, NRPSs, and hybrids of the two, 18 different possibilities arose for the combination of modules. A complete set of modules is included in Table 2, below, and FIG. 9. These 18 different possibilities generate a design space of 18 different modular megasynthases and the corresponding metabolites across four classes: PKSs, NRPSs, and two different hybrid classes, hybrids with one PKS module and two NRPS modules as well as hybrids with two PKS modules and one NRPS module (FIG. 9).

TABLE 2

| | | | | Exemplary |
|---|---|---|---|---|
| Modular | | Length of exemplary | Length of exemplary | Polynucleotide and |
| Megasynthase | Name | polynucleotide | polypeptide | Polypeptide Sequence |
| PKS | Ac-Mal_{H}-Mal_{OH} | 12.939 | 4.312 | SEQ ID NO: 108 |
| | | | | SEQ ID NO: 109 |
| PKS | Ac-Mal_{OH}-Mal_{OH} | 11.286 | 3.761 | SEQ ID NO: 110 |
| | | | | SEQ ID NO: 111 |
| PKS | Ac-Mal_{H}-Mal_{H} | 14.592 | 4.863 | SEQ ID NO: 112 |
| | | | | SEQ ID NO: 113 |
| PKS | Ac-Mal_{OH}-Mal_{H} | 10.029 | 4.312 | SEQ ID NO: 114 |
| | | | | SEQ ID NO: 115 |
| PKS(2)-NRPS(1) | Ac-Ser-Mal_{OH} | 10.029 | 3.342 | SEQ ID NO: 116 |
| Hybrid | | | | SEQ ID NO: 117 |
| PKS(2)-NRPS(1) | Ac-Ser-Mal_{H} | 11.682 | 3.893 | SEQ ID NO: 118 |
| Hybrid | | | | SEQ ID NO: 119 |
| PKS(2)-NRPS(1) | Ile-Mal_{OH}-Mal_{OH} | 11.598 | 3.865 | SEQ ID NO: 120 |
| Hybrid | | | | SEQ ID NO: 121 |
| PKS(2)-NRPS(1) | Ile-Mal_{OH}-Mal_{H} | 13.251 | 4.416 | SEQ ID NO: 122 |
| Hybrid | | | | SEQ ID NO: 123 |
| PKS(2)-NRPS(1) | Ile-Mal_{H}-Mal_{H} | 14.904 | 4.968 | SEQ ID NO: 124 |
| Hybrid | | | | SEQ ID NO: 125 |
| PKS(2)-NRPS(1) | Ile-Mal_{H}-Mal_{OH} | 13.251 | 4.416 | SEQ ID NO: 126 |
| Hybrid | | | | SEQ ID NO: 127 |
| PKS(2)-NRPS(1) | Ac-Mal_{OH}-Ser | 10.044 | 3.347 | SEQ ID NO: 128 |
| Hybrid | | | | SEQ ID NO: 129 |
| PKS(2)-NRPS(1) | Ac-Mal_{H}-Ser | 11.697 | 3.898 | SEQ ID NO: 130 |
| Hybrid | | | | SEQ ID NO: 131 |
| PKS(1)-NRPS(2) | Ile-Ser-Mal_{OH} | 10.341 | 3.446 | SEQ ID NO: 132 |
| Hybrid | | | | SEQ ID NO: 133 |
| PKS(1)-NRPS(2) | Ile-Ser-Mal_{H} | 11.994 | 3.997 | SEQ ID NO: 134 |
| Hybrid | | | | SEQ ID NO: 135 |
| PKS(1)-NRPS(2) | Ac-Ser-Ser | 8.787 | 2.928 | SEQ ID NO: 136 |
| Hybrid | | | | SEQ ID NO: 137 |
| PKS(1)-NRPS(2) | Ile-Mal_{OH}-Ser | 10.356 | 3.451 | SEQ ID NO: 138 |
| Hybrid | | | | SEQ ID NO: 139 |
| PKS(1)-NRPS(2) | Ile-Mal_{H}-Ser | 12.009 | 4.002 | SEQ ID NO: 140 |
| Hybrid | | | | SEQ ID NO: 141 |
| NRPS | Ile-Ser-Ser | 9.099 | 3.032 | SEQ ID NO: 142 |
| | | | | SEQ ID NO: 143 |

In one exemplary model library, a target library of possible combinations can be generated, as illustrated in FIGS. 1 and 2, which represents a strategy for combinatorial assembly of barcoded polyketide synthases (PKSs), non-ribosomal peptide synthases (NRPSs), and/or PKS-NRPS hybrids. Loading modules include a PKS and NRPS (Malonyl-CoA and Ile specific). Extension modules can include a KSAT-KR-ACP, KS-AT-DH-ER-KR-ACP (both Malonyl-CoA specific), C-A-PCP, and Cy-A-PCP (both Ser specific). This exemplary target library can be used to produce at least the 45 small molecules illustrated in the example below.

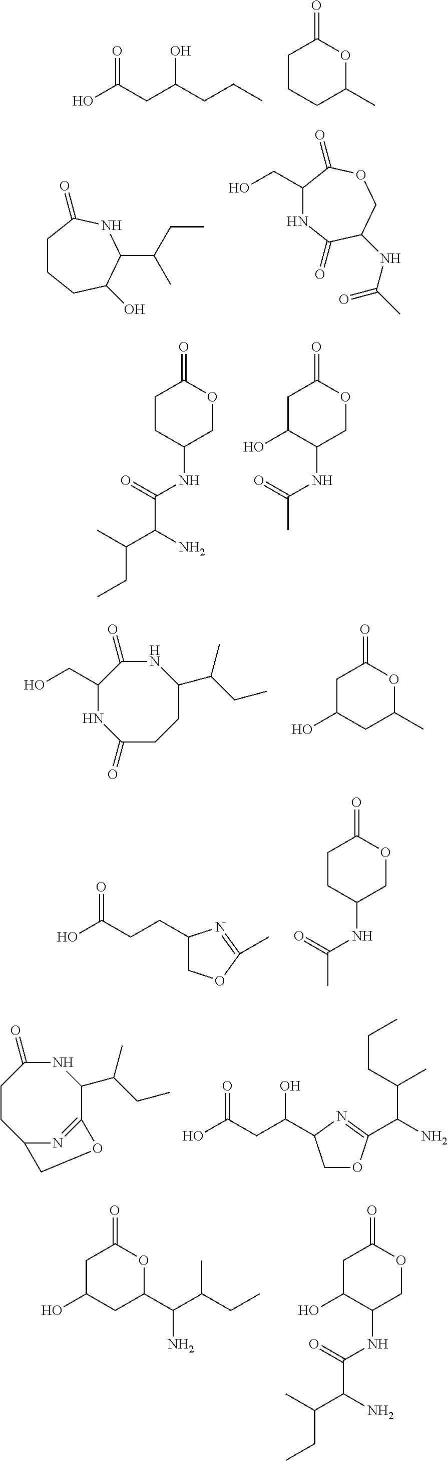

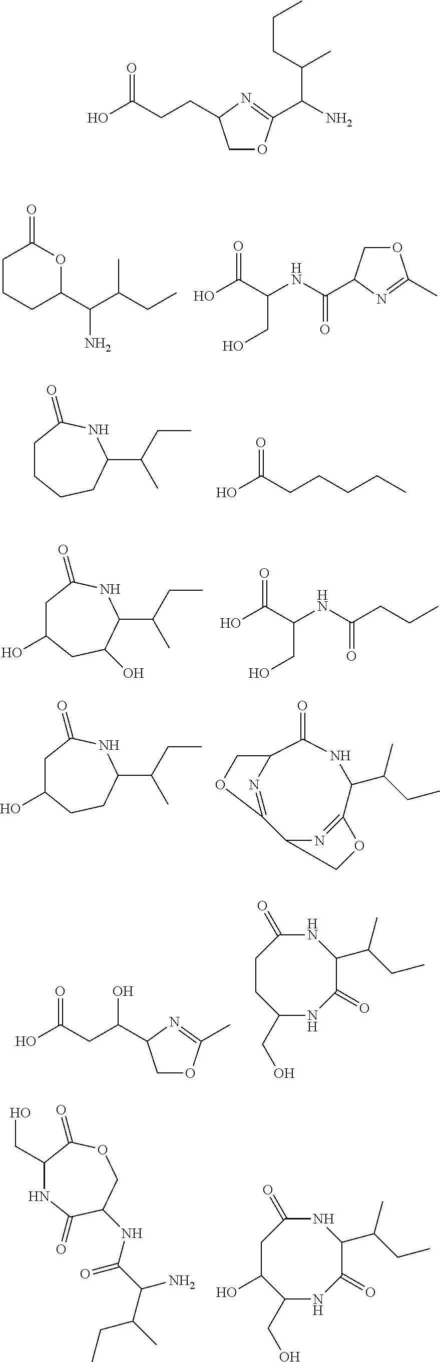

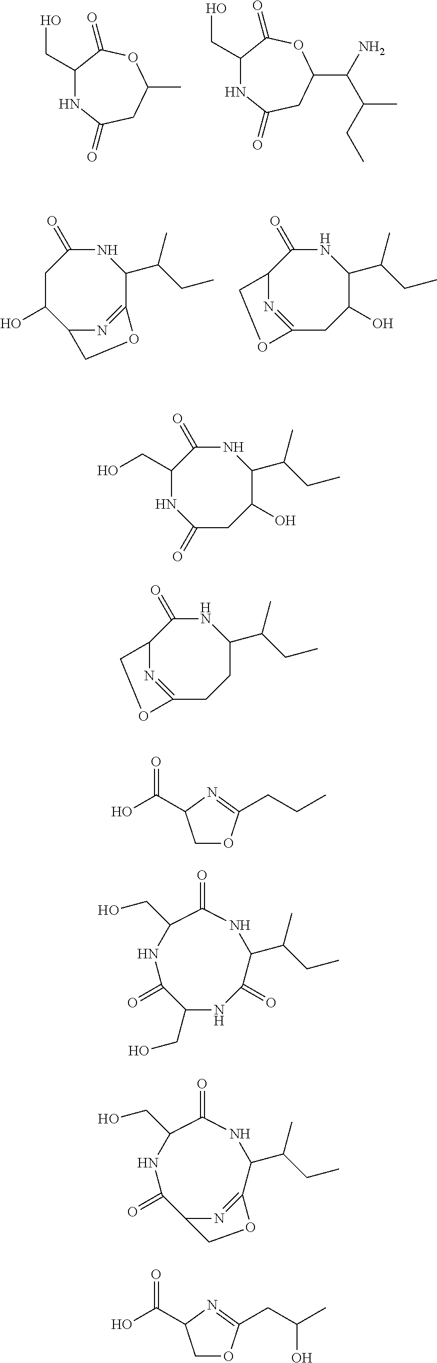

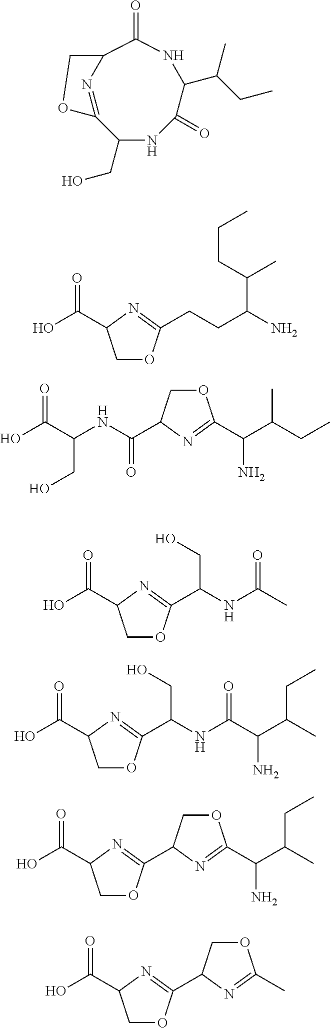

### Example 5

As disclosed previously, a wild-type gene of a naturally-occurring megasynthase is difficult to modulate, mutate or edit using in vivo editing tools such as CREATE. However, once the synthetic megasynthase has been successfully expressed in E. coli, by methods disclosed herein, targeted and random modifications are possible, as illustrated in FIGS. 13A-C.

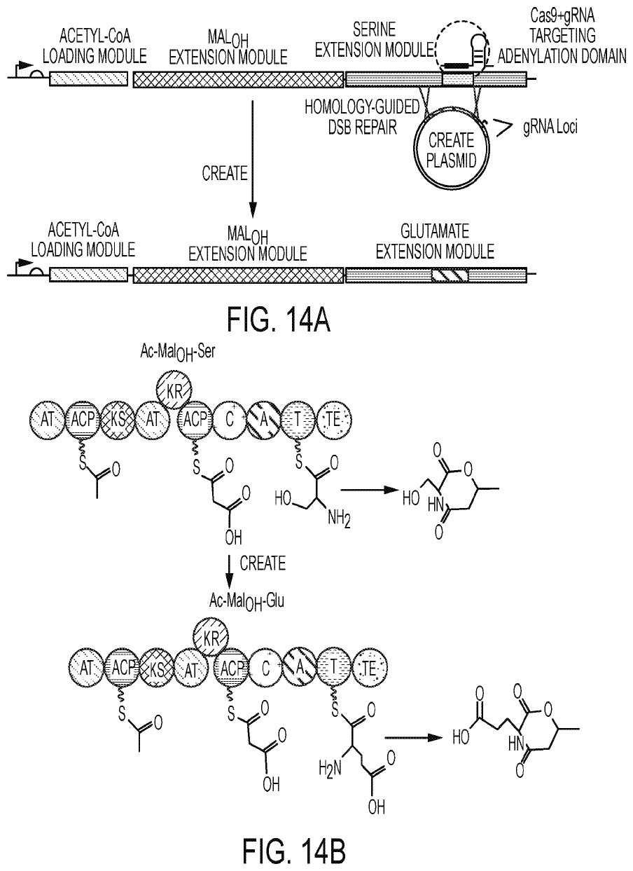

In one example, an extant Ac-Mal_{OH}-Ser construct described in Example 3 was synthesized and expressed in E. coli. In order to synthesize a different target molecule, one might envision needing to begin the entire process from the beginning. However, the serine-specific extension domain of the synthetic megasynthase was instead modified in vivo. Here, gene editing was used, using a CREATE plasmid inserted to replace the serine extension module with a glutamate extension module (FIG. 14A). With this change, the final synthetic product is not the 3-(hydroxymethyl)-7-methyl-1,4-oxazepane-2,5-dione synthesized by the Ac-Mal_{OH}-Ser megasynthase, but now is 3-(7-methyl-2,5-dioxo-1,4-oxazepan-3-yl)propanoic acid (FIG. 14B). In this method, using CREATE to edit the serine extension module in the Ac-Mal_{OH}-Ser synthetic megasynthase, at least 21 small molecules can be synthesized from the starting Ac-Mal_{OH}-Ser, as illustrated below:

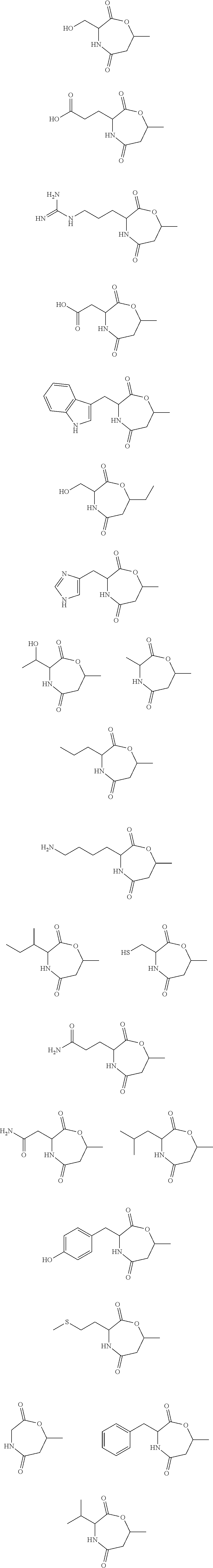

Using this approach, via genome editing using CREATE enables rapid construction of a large library of targeted small molecules.

### Example 6

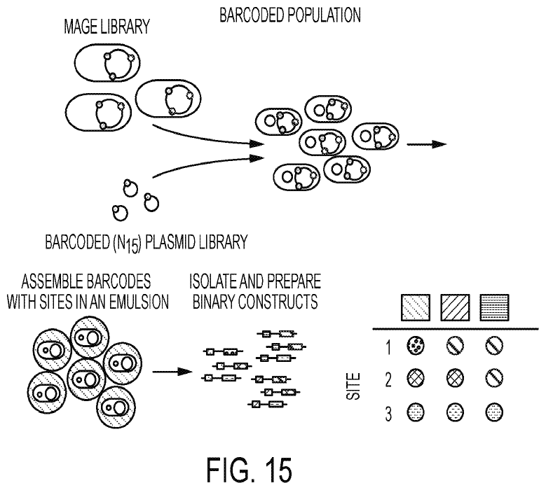

Exemplary methods described herein can be used to easily screen large libraries. FIG. 13A represents schematic diagrams illustrating CRISPR Enabled Trackable Genome Engineering (CREATE) cassette and design. CREATE vector contains both gRNA and an editing cassette in a size compatible with oligonucleotide chip synthesis. FIG. 13B represents Protospacer Adjacent Motif (PAM) mutation and editing. The CREATE editing cassette introduces a silent PAM mutation that protects from CRISPR cutting, coupled to the target mutation. FIG. 13C illustrates a CREATE strategy. CREATE cassettes are synthesized and cloned in a massively multiplexed fashion, allowing for massively multiplexed recombineering. A CREATE library can be designed to alter AT and A domain specificities, expanding the biosynthesis library from 32 to >10000 members. FIG. 15 illustrates barcoded-Tracking Combinatorial Engineering (bTRACE). Each member of the library is barcoded, and using multiplex linking PCR, various characteristics of each gene (i.e., module types and specificities) can be assembled to the barcode. These assembled constructs are MiSeq compatible. Once qualitative characteristics of the library are connected to barcodes, more quantitative data can be collected by sequencing just the HiSeq compatible barcodes.

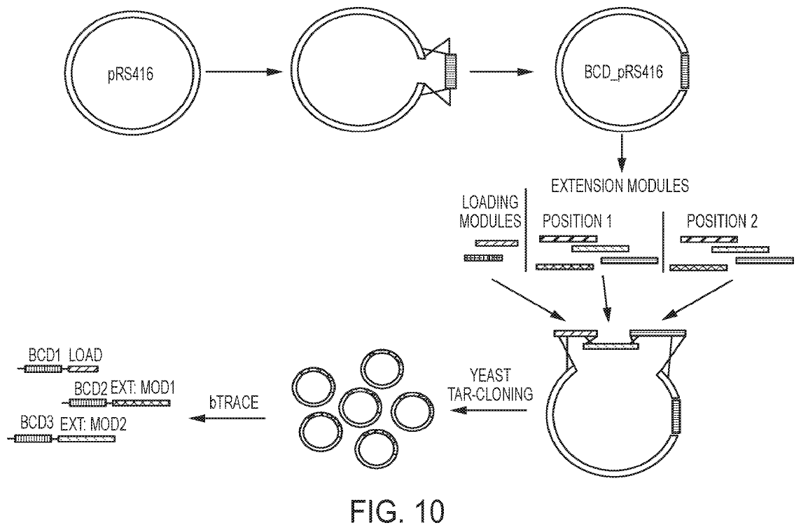

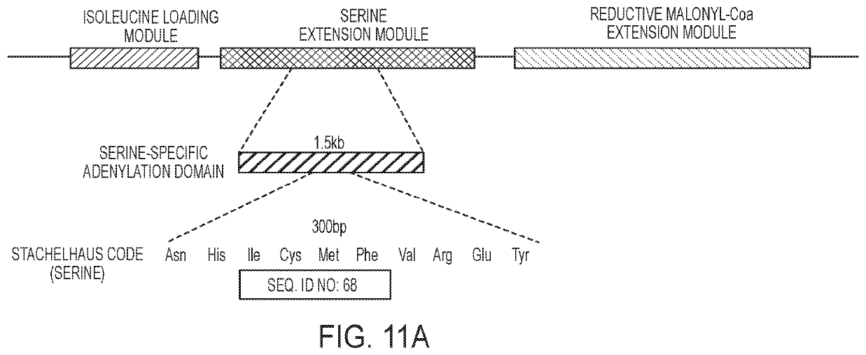

Typical methods used to screen such libraries involve demultiplexing each library member, scaling up production, and then screening, making a large labor intensive and expensive undertaking. By applying the techniques herein described, barcoded PKSs, NRPSs, and hybrids of the two can be created (FIG. 10), leading in turn to the creation of libraries of small molecules (FIGS. 14A and 14B). Because these combinatorial libraries are trackable (FIG. 15), screening acts as the demultiplexing step because this process would demultiplex leading directly to target compounds.

Screening against two different classes of diseases is described as an example. The underlying screening can be the same in both classes. It includes at least three parts: 1) a disease that is genetic in nature, 2) the causative mutation is in a protein that has a close homolog in E. coli, and/or 3) when the mutation is introduced into E. coli, it proves to be toxic under specific conditions.

### Example 7

In another exemplary method, screening of a loss-of-function genetic condition can be evaluated. For example, one loss-of-function genetic condition known as galactosemia is described. Galactosemia is caused by a mutation in a protein known as galT, preventing subjects having this mutation from metabolizing galactose. For reference, galactose is a common carbohydrate that is produced by the body as a by-product during metabolism of lactose. Galactosemia is fatal in 75% of infants having this trait, with symptoms such as an enlarged liver, cirrhosis, renal failure, cataracts, vomiting, seizure, hypoglycemia, lethargy, brain damage, and ovarian failure. The occurrence of galactosemia is about 1:60,000, making it extremely rare. The galT gene in humans is highly homologous to the galT gene required for E. coli to metabolize galactose. The galT gene in E. coli can be replaced with a human homolog, and no phenotypic differences will be observed. In addition, the most common mutation causing galactosemia prevents E. coli from using galactose as its sole carbon source and the organism parish when only galactose is provided. The screen for a galactosemia drug could involve combining the instant biosynthetic library with the mutated galT, inducing production of small molecules to create a library, and then selecting for E. coli growth on galactose. If E. coli survives, a small molecule that rescues function of the mutated galT can be produced, analyzed, and obtained for further study.

In an additional exemplary method, screening against a gain-of-function mutation in Kch, a voltage gated potassium pump, can be carried out per the disclosure herein. A specific mutation in the potassium sensing domain of Kch renders the pump overactive and is implicated in both heart conditions and epilepsy. When introduced into E. coli the mutation eliminates E. coli's ability to grow in media containing potassium. The screen for a mutation in Kch to remedy this condition could involve combining a biosynthetic library, as detailed herein, with a mutated Kch, inducing production of small molecules to create a library, and then selecting for E. coli growth on media having potassium. Under such conditions, if E. coli survives, a small molecule that rescues function of the mutated Kch permitting growth on media having potassium was produced in the mutant E. coli, and can be analyzed and obtained for further study and potential use in subjects having this disorder.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as can be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A construct, comprising:
a non-naturally occurring polypeptide having a formula represented by (X—B)ₙ—Z, wherein:
X comprises at least one polypeptide encoding at least one domain of a first target protein;
Z comprises at least one polypeptide encoding at least one domain of a second target protein;
B comprises a polypeptide for linking X and/or Z; and
n is a number from 1 to 100;
wherein the non-naturally occurring polypeptide having (X—B)ₙ—Z as a formula is capable of synthesizing a secondary metabolite in a cell or organism.

2. The construct according to claim 1, wherein the at least one domain comprises at least one catalytic of the first target protein or the second target protein.

3. The construct according to any one of the preceding claims, wherein the first target protein comprises a megasynthase, a polyketide synthase, a non-ribosomal peptide synthase, and/or hybrid thereof.

4. The construct according to any one of the preceding claims, wherein the second target protein comprises a megasynthase, a polyketide synthase, a non-ribosomal peptide synthase, and/or hybrid thereof.

5. The construct according to any one of the preceding claims, wherein the construct comprises a modular megasynthase.

6. The construct according to any one of the preceding claims, wherein the first target protein and the second target protein are the same protein.

7. The construct according to any one of the preceding claims, wherein X comprises a polypeptide sequence of at least one domain selected from the group consisting of: Acyltransferase (AT), Acyl Carrier Protein (ACP), Keto-Synthase (KS), Ketoreductase (KR), Dehydratase (DH), Enoylreductase (ER), Methyltransferase (MT), Sulfhydrolase (SH), and Thioesterase (TE).

8. The construct according to any one of the preceding claims, wherein Z comprises a polypeptide sequence of at least one domain selected from the group consisting of: Acyltransferase (AT), Acyl Carrier Protein (ACP), Keto-Synthase (KS), Ketoreductase (KR), Dehydratase (DH), Enoylreductase (ER), Methyltransferase (MT), Sulfhydrolase (SH), and Thioesterase (TE).

9. The construct according to any one of the preceding claims, wherein B comprises a polypeptide sequence selected from the group consisting of: Acyl Carrier Protein-Condensation Domain linkers (ACP Condensation), Acyl Carrier Protein-Heterocyclization Domain linkers (ACP Heterocyclization), Acyl Carrier Protein-Ketosynthase Domain linkers (AC-PKS), Acyl Carrier Protein-Thioesterase Domain linkers (ACP-TE), Adenylation Domain-Peptide Carrier Protein linkers (A-PCP), Acyltransferase Domain-Acyl Carrier Protein linkers (AT-ACP), Acyltransferase Domain-Dehydratase Domain linkers (AT-DH), Acyltransferase Domain-Ketoreductase Domain linkers (AT-KR), Condensation Domain-Adenylation Domain linkers (Condensation A), Dehydratase Domain-Enoylreductase Domain linkers (DH-ER), Dehydratase Domain-Ketoreductase Domain linkers (DH-KR), Dual Condensation/Epimerization Domain-Adenylation Domain linkers (Dual Condensation A), Enoylreductase Domain-Ketoreductase Domain linkers (ER-KR), Heterocyclization Domain-Adenylation Domain linkers (Heterocyclization A), (Both Acyl and Peptide) Carrier Protein-Condensation Domain linkers (Joint AC-PC), Ketoreducatse Domain-Acyl Carrier Protein linkers (KR-ACP), and Ketosynthase Domain-Acyltransferase Domain linkers (KS-AT).

10. The construct according to any one of the preceding claims, wherein the secondary metabolite comprises:

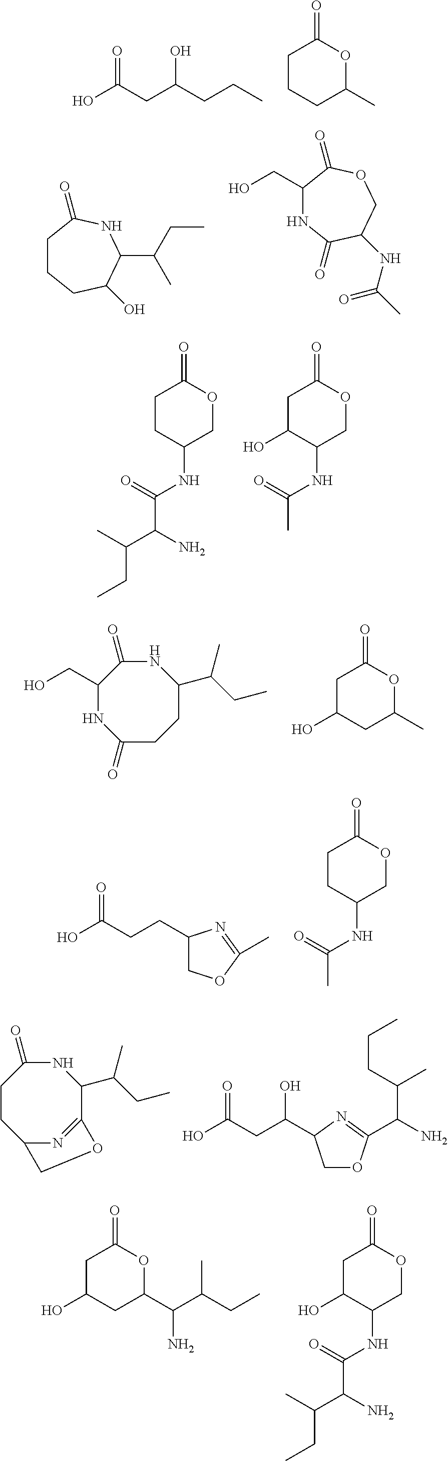

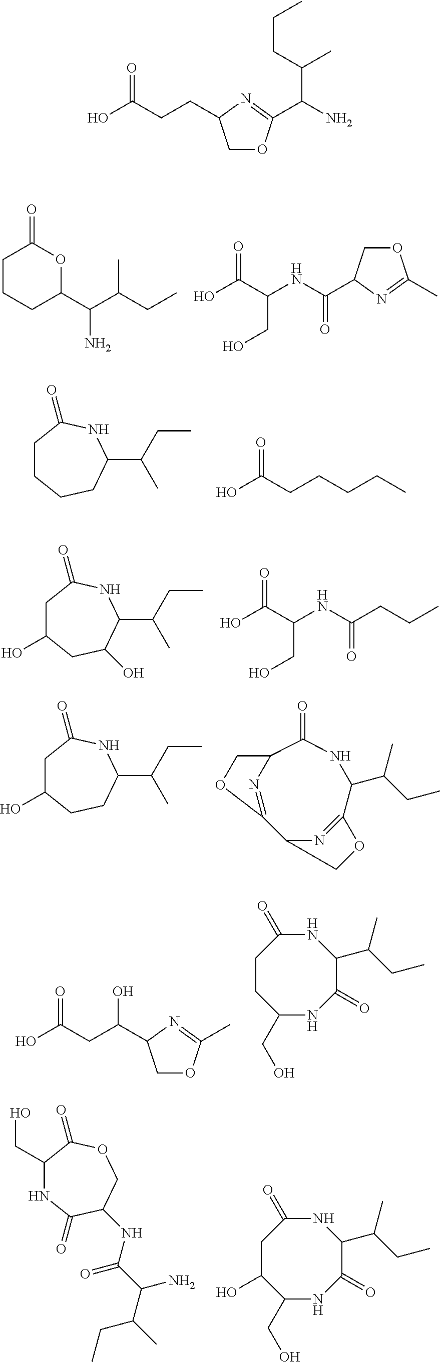

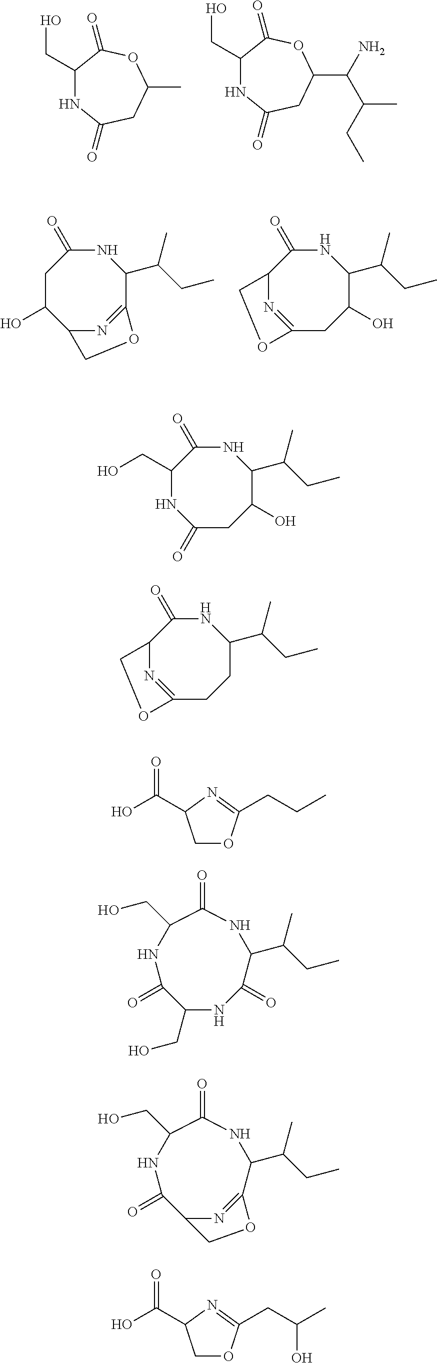

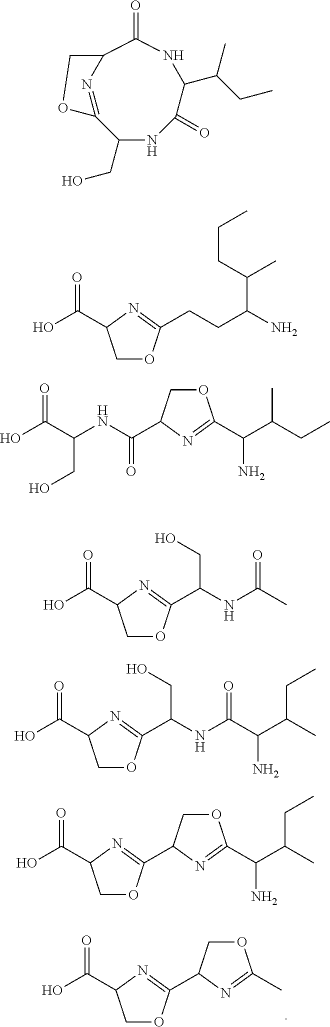

11. The construct according to any one of the preceding claims, wherein the secondary metabolite is selected from the group consisting of: delta-hexalactone, Rapanycin, Actinorhodin, Erythromycin A, 6-Methylsalicyclic acid, Aflatoxin B1, Rifamycin S, Lovastatin, Amphotericin B, and Monensin A.

12. The construct according to any one of the preceding claims, wherein the first target protein or the second target protein is a prokaryotic protein or a eukaryotic protein.

13. The construct according to any one of the preceding claims, the construct having the formula (X—B)ₙ—Z is a polypeptide having at least 95 percent identity to at least one polypeptide selected from the group consisting of the polypeptides represented by at least one of SEQ ID NOs: 33-64, 109, 111, 113, 115, 117, 119, 121, 123, 125, 127, 129, 131, 133, 135, 137, 139, 141, 143.

14. The construct according to any one of the preceding claims, wherein B comprises a polypeptide sequence represented by at least one of SEQ ID NOs: 70-71, 73-74, 76-79, 81-82, 84-86, 88-91, 93-96, 98-99, 101-107.

15. A non-naturally occurring polynucleotide comprising a polynucleotide encoding the construct of any one of the preceding claims.

16. The polynucleotide according to any one of claim 15, wherein the polynucleotide can be compiled together with other polynucleotides to make a library of one or more target proteins, secondary metabolites or a trait.

17. The polynucleotide according to claim 15 or 16, wherein the polynucleotide is a polynucleotide having at least 95 percent identity to at least one polynucleotide selected from the group consisting of the polynucleotides represented by at least one of SEQ ID NOs: 1-32, 108, 110, 112, 114, 116, 118, 120, 122, 124, 126, 128, 130, 132, 134, 136, 138, 140, 142.

18. A host for producing a construct according to any one of claims 1-14.

19. The host according to claim 18, wherein the host comprises Escherichia coli.

20. The host according to claim 18, wherein the construct produces a genetic modification to overcome a deleterious trait in a human gene where the human gene is homologous to an E. coli gene and wherein the deleterious trait is selectable.

21. A method for generating a construct, comprising:
obtaining at least one polynucleotide sequence encoding at least one domain of a first target protein and at least one polynucleotide sequence encoding at least one domain of a second target protein;
determining a linker polynucleotide that is capable of encoding a protein linking the at least one domain of the first target protein and the at least one domain of the second target protein; and
generating a polypeptide construct having the at least one polypeptide sequence encoding the first target protein and at least one polypeptide sequence encoding the second target protein and a polypeptide sequence encoding the linker polypeptide.

22. The method according to claim 21, wherein the step of determining the polynucleotide sequence encoding the linker polypeptide further comprising creating a gene cluster annotation of the target gene encoding the linker polypeptide.

23. The method according to any one of claims 21-22, further comprising converting the construct having the at least one polypeptide sequence and the linker sequence into at least one nucleotide sequence by using codon harmonization.

24. A method for generating a biopharmaceutical agent, comprising:
introducing into cells a vector that encodes a construct according to any one of claims 1-14;
obtaining viable cells expressing the vector; and
isolating the biopharmaceutical agent from the viable cells.

25. The method according to claim 24, further comprising: introducing a mutation into the cells, wherein the mutation causes a condition.

26. The method according to claim 25, wherein the condition comprises a genetic disease.

27. The method according to claim 26, wherein the genetic disease comprises a loss-of-function genetic disease or a gain-of-function genetic disease.

28. A method for generating an in vivo construct library comprising generating the polynucleotides according to claims 15 to 17, wherein each polynucleotide represents one genetic variation in a target gene of a target protein and the construct library comprises all naturally-occurring and non-natural amino acid residue changes of the target protein.

29. A method comprising:
assigning ranks pertaining to biological effects of genetic variations of a plurality of genes or genetic loci capable of coding for a target protein;
assigning ranks pertaining to the biological effect due to the genetic variations of the plurality of genes or genetic loci;
obtaining and analyzing one or more rank(s) of the genetic variations of the genes or genetic loci pertaining to a predetermined selection process;
obtaining one or more composite rank(s) based on the ranks of the biological effects as they pertain to the predetermined selection process and biological context rank; and
designing a genomically-engineered process, cell or organism based on the composite rank(s).

30. The method according to claim 29, wherein the biological effect comprises a modulation of the target gene.

31. The method according to claim 30, wherein the target gene comprises an enzyme and the modulation of the target gene comprises an increase in biological activity of the enzyme compared to a target gene not having the genetic variation.

32. The method according to claim 29, where the assigning comprises measuring the effect of the genetic variation on a specific trait.

33. A computer-readable medium having computer-readable instructions, which, when executed by a computer, cause the computer to carry out a method comprising:
receiving first gene(s) or genetic segment score representing a score of a biological effect or condition due to a genetic variation of a gene or gene segment of a target protein;
receiving at least a second gene(s) or genetic score representing a second score of another genetic variation of the target protein;
combining the scores; and
assigning a combined score related to one or more genetic variations in order to assess a value of the genetic variations related to a trait for the target protein.

34. The computer-readable medium of claim 33, further comprising designing a genomically-engineered organism or cell based on the composite scores for two or more genes or genetic loci.

35. The computer-readable medium of claim 33, wherein information related to more than one target gene can be received and assessed.

36. A system comprising:
a component for assessing a score of a genetic variation of genes or genetic segments pertaining to a trait of one or more target proteins; and
a component for reporting the score of the genetic variation of genes or genetic segments pertaining to a trait of one or more target proteins; and
a component for compiling the scores of one or more target proteins.

37. The system according to claim 36, wherein the genetic variation comprises a mutation, insertion, deletion or other genetic variation.

38. A library comprising the constructs of claims 1-14 and/or the polynucleotides of claims 15-17.

39. The library according to claim 38, wherein the library is a genomic library of a target microorganism.

40. The library according to any one of claims 38-39, wherein the constructs comprise all possible genetic variations together in a pool representing every mutated residue of the target protein.

41. A method for engineering a polypeptide construct comprising:
obtaining the polynucleotide according any one of claims 15 to 17;
obtaining one or more oligonucleotide sequences, each containing regions of homology to one or more target gene(s), and regions of genetic variation towards one or more target gene(s);
using the one or more oligonucleotide sequences to generate amplified constructs comprising regions of homology suitable for homologous recombination within the polynucleotide; and
using the amplified constructs to create a library of mutant target genes within the polypeptide construct.

42. The method according to claim 41, further comprising a traceable barcode positioned outside of the gene or the gene segment open reading frame in the amplified constructs, wherein the traceable barcode corresponds to or is quantitatively linked to a genetic variation of the gene or the gene segment.
